(19) United States
(12) Patent Application Publication
(10) Pub. No.: US 2025/0057447 A1
(43) Pub. Date: Feb. 20, 2025

(54) MULTISPECTRAL IMAGING ASSESSMENT METHOD FOR GAIT DISORDERS AND STATIC PRESSURE HYDROTHERAPY EXERCISE DEVICE

(71) Applicants: Chi-Hseng Hsieh, Hsinchu City (TW); Szu-Yuan Chen, Taipei (TW)

(72) Inventors: Chi-Hseng Hsieh, Hsinchu City (TW); Szu-Yuan Chen, Taipei (TW)

(21) Appl. No.: 18/233,882

(22) Filed: Aug. 15, 2023

(51) Int. Cl. A61B 5/11; A61B 5/00; G06T 7/00

(52) U.S. Cl. CPC A61B 5/112; A61B 5/004; A61B 5/0062; A61B 5/7405; G06T 7/0012; A61B 2505/09; A61B 2560/0406; A61B 2576/02

(57) ABSTRACT

An evaluation method for gait-impaired individuals, including freezing gait in Parkinson's disease, and a corresponding hydrostatic water therapy pool, is provided. A multispectral thermal imager is utilized to observe and record the multispectral images of COP (Center of Pressure) trajectories in both on-land and underwater scenarios on black walkways. These images, including far/near-infrared and visible light with gait temporal/spatial parameters, serve as references for analysis and interpretation by physical therapists and attending physicians. Additionally, a personalized hydrostatic water therapy pool rehabilitation program is provided in conjunction with the patient or elderly individual, establishing a correlation between the evaluation and training. This approach enhances the effectiveness of evaluation and analysis, reduces healthcare costs, mitigates psychological barriers related to fear of falling during exercise, and ultimately improves the health and quality of life for individuals with gait-impaired individuals.

### FIELD OF THE DISCLOSURE

The present invention relates to a hydrotherapy system, specifically focusing on a multispectral imaging evaluation method for gait-impaired individuals and its corresponding hydrotherapy exercise device incorporating static water pressure.

### BACKGROUND OF THE INVENTION

Gait disorders significantly impact the quality of life for patients. The elderly with gait disorders has a higher mortality rate compared to those without such disorders. Additionally, individuals who suffer from other diseases along with gait abnormalities experience an even greater increase in mortality rate. This demonstrates the compounding detrimental effect of gait disorders in conjunction with other illnesses. The main causes of gait disorders in clinical settings include sensory deficits, myelopathy (spinal cord disorders), and Parkinson's disease (referred to as PD).

Under traditional clinical conditions, the analysis of gait parameters by experts may sometimes lack the required accuracy, leading to negative impacts on pathological diagnosis. In this case, the invention aims to improve the evaluation efficiency and provide experts with a device and technology capable of objectively evaluating different gait parameters using multispectral imaging. This evaluation includes monitoring and comparing processes both on “solid ground” and “underwater.” By doing so, a significant amount of reliable and correlated information regarding the gait of patients (or fellow patients) is provided, thereby reducing the tolerance for errors caused by subjective techniques.

The clinical evaluation of gait disorders generally categorizes the severity into three approximate levels for the sake of convenience: mild, moderate, and severe. Mild cases may involve joint, muscle, or sensory impairments, resulting in sensorimotor coordination issues. Moderate cases are often caused by abnormalities in the motor nervous system, including Parkinson's disease (referred to as PD), as emphasized in this case. Severe gait disorders can originate from both cerebral and psychological factors.

In a typical case of Parkinson's disease, patients typically exhibit slow walking speed, small steps with a high step frequency, involuntary leaning forward or backward, and other symptoms such as a masked facial expression, reduced blinking, stooped posture, and drooling. If left untreated, late-stage Parkinson's disease may be accompanied by depression and dementia. Therefore, patients often exhibit a shuffling gait, which is characterized by obvious irregularities and can significantly impact their functional independence and quality of life.

Parkinson's disease is a chronic neurodegenerative disease that seriously affects the health of middle-aged and elderly individuals. Currently, the treatment of Parkinson's disease focuses mainly on symptom “management”. The diagnosis of Parkinson's disease relies on medical history and neurological examinations, as there is currently no definitive test to confirm the disease. A complete cure for Parkinson's disease has not been developed yet, but there are medications, surgeries, and interdisciplinary integrated therapies available that can help “alleviate” the symptoms.

In the general medical field, there are three main principles for assessing gait disorders: (1) Consideration of gait disorders arising from pain and fear of falling, such as the occurrence of freezing of gait (FOG) phenomenon; (2) Consideration of factors related to lower limb instability, including muscle strength in the legs. Patients may experience dragging of the feet, slow walking speed, short stride, stiffness, reduced step height, and hesitant gait. Symptoms improve with assistance; (3) Consideration of observing the stride length of the same foot, step width compared to the other foot, symmetry of both feet, as well as the variability range of step width or foot patterns.

Regarding the aforementioned three principles for assessing gait disorders, this case proposes a corresponding static pressure hydrotherapy exercise device. This is because the buoyancy in water reduces the fear of falling, and even if the patient does stumble, the risk of injury is minimal.

In terms of considering factors related to limb instability and the variability range of gait or foot patterns, it is combined with the inventor's previous invention under Taiwanese patent TWI666935 titled “System and Method for Gait Footprint Analysis Based on Sigma-Type Multispectral Imaging”. This provides a safe, normal, economical, and efficient evaluation and analysis, along with a corresponding static water therapy approach.

Freezing of gait (FOG) occurs in approximately 50% of Parkinson's disease patients in the later stages. It is characterized by sudden, transient episodes of gait cessation. It often happens during turning, passing through narrow pathways, multitasking, or approaching a target.

According to data from the Parkinson's Foundation, nearly one million Americans are affected by Parkinson's disease. Globally, the United States diagnoses approximately 60,000 new cases each year. In addition to diagnosed cases, thousands of Americans unknowingly have Parkinson's disease. The foundation states that some Parkinson's patients turn to “aquatic therapy” as a choice to improve their quality of life and overall health.

Aquatic therapy remains a widely recognized treatment method applicable to various diseases. It can assist patients with cardiovascular conditions, mild injuries, and Parkinson's disease, among other serious illnesses. Even individuals with general good health and high endurance athletes turn to this form of therapy to enhance endurance and facilitate the recovery from various injuries.

In Omaha, Nebraska, there is a rehabilitation center that offers an Aquatic Therapy Program encouraging Parkinson's patients to participate. Aquatic therapy refers to any exercise or therapy conducted in a controlled and monitored water environment, typically a swimming pool. Water-based activities in a heated pool can improve overall health and well-being through exercise. Aquatic therapy involves physical therapists implementing therapeutic programs in a swimming pool or water environment.

Based on the buoyancy phenomenon of water, it can counterbalance approximately 80-90% of body weight. Therefore, a person weighing 100 kg would feel as if they weigh only 10-20 kg while in water. This buoyancy assistance allows for greater movement, enabling Parkinson's patients to practice walking with more normal or exaggerated gait patterns and gradually engage in larger range movements. Meanwhile, the resistance and turbulence challenge balance and coordination. Their movements in water require less effort and are more relaxed compared to performing the same movements on land.

In present, we can utilize video recording, gait analysis systems, foot pressure testing, and dynamic thermographic imaging to further quantitatively in analyze footprints, including temporal and spatial parameters, to assess the gait function and quality of movement in Parkinson's patients.

For instance, in Taiwanese patent TWI657800 titled “Method and System for Gait Analysis”, it discloses a method that involves the use of multiple acceleration sensors. The method includes calculating the square root based on the acceleration values sensed by each acceleration sensor along the sensing axis at each time point. It further involves calculating the cross-correlation coefficient based on the square roots of the first and second acceleration sensors, calculating the first autocorrelation coefficient of the square root of the first acceleration sensor, calculating the second autocorrelation coefficient of the square root of the second acceleration sensor, and finally, computing the first gait index based on the cross-correlation coefficient, first autocorrelation coefficient, and second autocorrelation coefficient.

According to Taiwanese patent TWI637738 titled “Wearable Walking Assistance Device and Walking Assistance Method’, it discloses a wearable walking assistance device that provides users with feedback information regarding the road conditions ahead. The device includes: a wearable garment designed for users to wear; multiple distance sensors positioned at various locations on the wearable garment; a processor connected to these distance sensors, utilizing the distance sensing information provided by them, which includes data on different heights, horizontal angles, vertical angles, and orientations, and the processor compares this information with stored data to obtain environmental information; a storage module connected to the processor, providing the stored data; a wireless module connected to the processor, enabling wireless connectivity to at least one monitoring end; a feedback module connected to the processor, providing feedback information based on the obtained environmental information.

According to Taiwanese patent TWI581829 titled “Smart Suspension System with Gait Analysis Function”, it discloses an invention applicable to general exercise devices or rehabilitation devices to assist users in rehabilitation training. The invention relates to a device applicable to general sports equipment or rehabilitation devices for assisting users in rehabilitation training. The device is equipped with a weight sensor and a displacement sensor in a position area capable of detecting and displaying changes in user weight and gait on the sports equipment. The sports equipment has a transmission mechanism connected to a vehicle that can restrain the user, allowing the user to be supported on the sports equipment through a smart suspension system. The support force can be appropriately adjusted by detecting the user's lower limb weight-bearing capacity through the weight sensor. Furthermore, the displacement sensor detects and analyzes the user's gait in real-time, providing immediate feedback. This visual feedback allows the user to understand the gait condition during exercise or rehabilitation and make appropriate gait adjustments. The feedback signal can also be used for operational control of the rehabilitation device.

According to Taiwanese invention patent TWI578961, titled “Thermosensitive Color-Changing Gait Analysis System,” a platform is provided for the subject to step and walk on, and corresponding color-changing regions are generated based on the temperature variations in each contact area of the foot sole during each step. An image capturing device captures images of the color-changing regions. A gait analysis device analyzes the shape and color changes of each color-changing region and establishes stepping pattern data for each corresponding foot sole. By analyzing the color variations generated by stepping on the platform, more accurate foot stepping information can be obtained, significantly improving the accuracy of the established gait data. It is an innovative and more accurate design for gait analysis system, characterized by a platform (3) with a transparent plate (31) and a thermosensitive color-changing film (32) fixed on the top surface of the plate (31). In this embodiment, the transparent plate (31) allows the color changes of the thermosensitive color-changing film (32) to be viewed from below.

Furthermore, other examples include Taiwanese invention patent TWI579530 titled “Mobile Device Step Counting System and Gait Analysis Method”, Taiwanese invention patent TWI549033 titled “Touch-Sensing Gait Analysis System”, Taiwanese invention patent TWI517875 titled “Gait Analysis Device and Application in Running Exercise Equipment”, and so on.

Additionally, as of the preparation of this invention, a search using keywords such as “Parkinson's disease” or “Parkinson” and “aquatic exercise therapy” or “hydrotherapy” was conducted on the Taiwanese Patent Information Retrieval System and related global patent retrieval systems.

Among them, in the evaluation of postural balance changes with aquatic therapy intervention, a search was conducted for all articles that meet the criteria, using the keywords “multi-spectral thermography, Parkinson's disease, and freezing gait”. However, there were no retrieval results found.

### SUMMARY OF THE INVENTION

The present invention relates to a hydrostatic water therapy system and an evaluation and analysis system designed for individuals with gait impairments. The hydrostatic water therapy system comprises an adjustable black walkway platform and a hydrostatic water therapy pool. The adjustable black walkway platform includes adjustable pillars, a black walkway, a multispectral thermal imager, and a safety unit. The safety unit may include a wide-bottom handrail, a weight-reducing sling, or a walker. The platform may also include a VC temperature control plate and its controller. The black walkway may have a test area and a scale table on both sides.

The hydrostatic water therapy pool includes a pool body, a water source device, and a window. The water source device is configured to perform functions including water circulation, replacement cleaning, disinfection sterilization, and hot and cold water supply and temperature control. The multispectral thermal imager includes a near-infrared auxiliary light source with 940 nm LEDs.

The hydrostatic water therapy system uses the adjustable black walkway platform and the hydrostatic water therapy pool to capture multispectral footprint images of the gait-impaired individual both inside and outside the pool, thereby achieving dual functionality of evaluation and corresponding exercise.

The evaluation and analysis system for gait-impaired individuals' footprints includes evaluation mode, evaluation method, evaluation subjects, evaluation items and images, visual and auditory guidance methods, and physical therapist coaching methods for evaluating the position of body center of gravity response. The system serves as the basis for taking relevant corresponding hydrostatic water therapy pool evaluation and analysis and exercise plan. The evaluation and analysis system is used to assess balance ability indicators of the gait-impaired individual's footprint COP area and movement trajectory, and assists in identifying the most effective exercise plan process.

The images used in the evaluation and analysis system include far-infrared with a wavelength range of 8-14 μm, near-infrared with a wavelength range of 0.8-1.0 μm, and visible light with a wavelength range of 0.4-0.8 μm, all three types of footprint images. The position of the body center of gravity response further includes a connection line of the body's COP and COG. The evaluation and analysis system may further include a hydrostatic water therapy pool, which includes warm water, a pool body, water source equipment, and a window. The material of the pool body may be strengthened thick transparent glass. The visible light may include the window image of the pool body.

The foregoing, as well as additional objects, features and advantages of the invention will be more readily apparent from the following detailed description, which proceeds with reference to the accompanying drawings.

### BRIEF DESCRIPTION OF THE DRAWINGS

The objects, spirits, and advantages of the preferred embodiments of the present disclosure will be readily understood by the accompanying drawings and detailed descriptions, wherein:

FIG. 1A is a schematic diagram of the adjustable black platform.

FIG. 1B is a schematic diagram of the principle of road surface evaluation and analysis.

FIG. 1C is a schematic diagram of the principle of underwater evaluation and analysis.

FIG. 1D is a schematic diagram of the VC temperature-changing plate.

FIG. 2A is a schematic diagram of the black walkway.

FIG. 2B is another schematic diagram of the black walkway.

FIG. 2C is a schematic diagram of the wide-base handrail.

FIG. 2D is a schematic diagram of footprint photography.

FIG. 2E is another schematic diagram of footprint photography.

FIG. 3A is a schematic diagram of the static pressure hydrotherapy pool.

FIG. 3B is a schematic diagram of the glass pool body.

FIG. 4A is a schematic diagram of near-infrared image characteristics.

FIG. 4B is a schematic diagram of the color palette of the multispectral thermal imager.

FIG. 5A is a schematic diagram of thermal images of the lower limbs.

FIG. 5B is a schematic diagram of a normal gait footprint.

FIG. 5C is a schematic diagram of a thermal gait footprint.

FIG. 5D is another schematic diagram of a thermal gait footprint.

FIG. 6A is a schematic diagram of freezing of gait.

FIG. 6B is another schematic diagram of freezing of gait.

FIG. 6C is a COP schematic diagram for Parkinson's disease.

FIG. 7A is a schematic diagram of the vertical line of body weight.

FIG. 7B is another schematic diagram of the vertical line of body weight.

FIG. 7C is another schematic diagram of the vertical line of body weight.

FIG. 7D is another schematic diagram of the vertical line of body weight.

FIG. 8A is a schematic diagram of guided exercise.

FIG. 8B is another schematic diagram of guided exercise.

FIG. 8C is another schematic diagram of guided exercise.

FIG. 9A is a schematic diagram of leg lifting exercise.

FIG. 9B is another schematic diagram of leg lifting exercise.

FIG. 10A is a schematic diagram of multispectral thermal imaging.

FIG. 10B is a schematic diagram of multispectral near-infrared imaging.

FIG. 11A is a schematic diagram of standing on the black walkway.

FIG. 11B is a schematic diagram of foot pattern.

FIG. 11C is a schematic diagram of standing stability.

FIG. 11D is a schematic diagram of the footprint image area.

FIG. 11E is a schematic diagram of the footprint parameters.

### DESCRIPTION OF THE PREFERRED EMBODIMENTS

In terms of the overall evaluation and analysis of gait evaluation and corresponding exercise methods for lower limbs, this invention addresses two main issues:
1. Regarding the evaluation and analysis of popular wearable sensors among patients, such as the “Whole-Body Gait Analysis System” currently disclosed by Taiwan's Industrial Technology Research Institute, it poses a significant challenge due to the requirement for multiple sensors to be worn on the entire body by elderly individuals or those with gait disorders. This poses a “deficiency.”
2. In the current aquatic exercise programs designed for individuals with gait disorders, such as hydrotherapy programs like the well-known HydroWorx treatment pool in the United States, they serve purely as exercise tools and fail to fulfill the requirements for evaluation and analysis.

To address the aforementioned deficiencies, this application proposes solutions that can resolve these issues and improve upon existing technologies.

### These Solutions Include:

(1) An evaluation and analysis method for elderly individuals or those with gait disorders that does not necessitate the use of multiple sensors worn on the entire body.
(2) An exercise tool that not only serves as a means of physical exercise for elderly individuals or those with gait disorders but also incorporates an evaluation and analysis method.

The proposed solution to contribute to (1) and (2) is the utilization of a multispectral imaging evaluation and analysis method in conjunction with a corresponding hydrotherapy system employing static pressure.

The technical solution proposed to address the problem brings significant advantages compared to existing technologies:
1. It can alleviate the economic burden and daily life pressures faced by gait-impaired individuals.
2. It can mitigate the fear of falling and the inconvenience associated with evaluation tests, as well as provide the advantage of immediate exercise following evaluation and immediate evaluation after exercise.

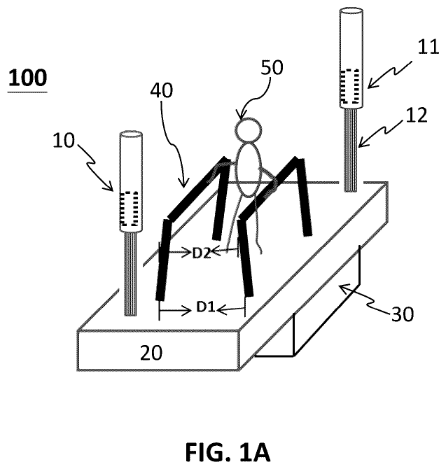

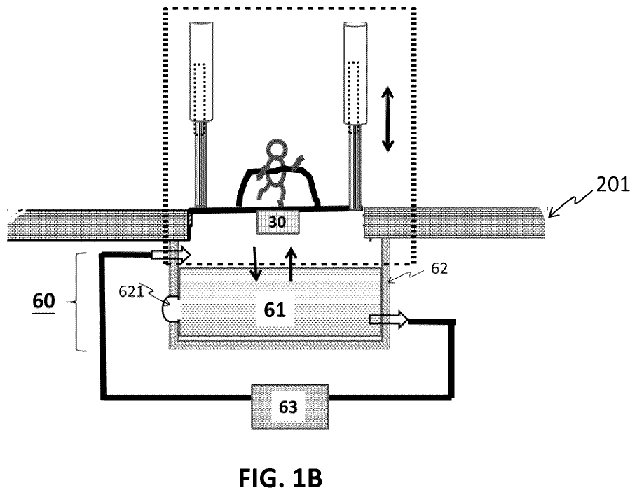

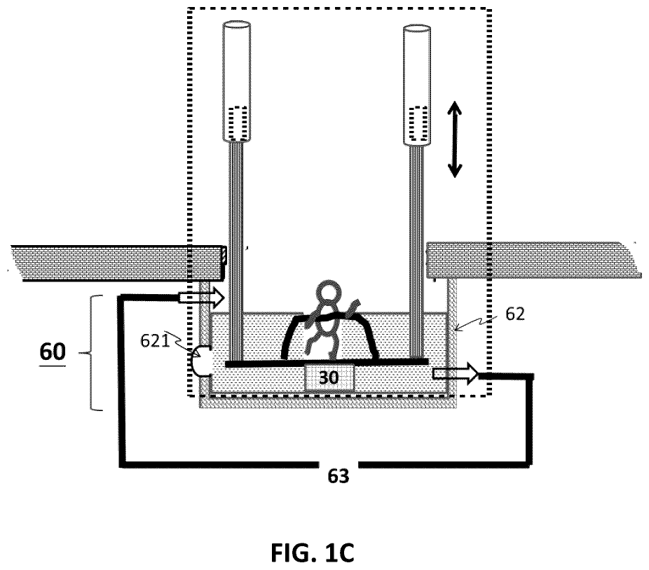

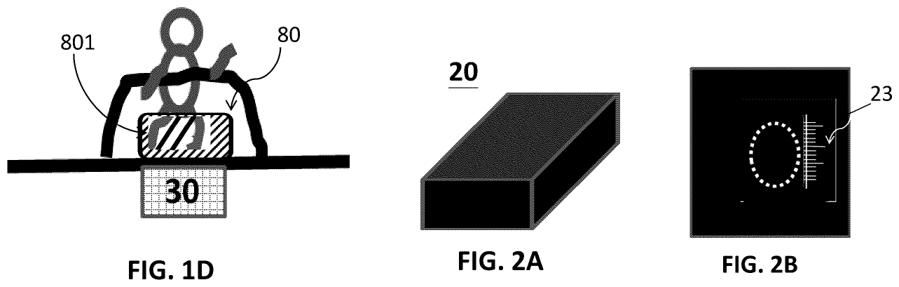

To facilitate the explanation of the basic principles and functioning mechanism of the invention, please refer to FIGS. 1, 1A, 1B, and 1C for a better understanding.

FIG. 1A illustrates a schematic diagram of an adjustable black walkway platform. FIG. 1B represents a schematic diagram of the evaluation and analysis principles on the ground surface. FIG. 1C depicts a schematic diagram of the evaluation and analysis principles underwater. FIG. 1D showcases a schematic diagram of the VC temperature-controlled plate.

In FIGS. 1A to 1D, the red lines are highlighted to emphasize the position of the individual (patient) 50 on the black walkway 20, aiming to provide clarity. FIG. 1A presents an adjustable black walkway platform 100 comprising adjustable pillars 10, a black walkway 20, a monitoring box 30, and a wide-based handrail 40. The patient 50 stands on the black walkway 20. In this embodiment, the black walkway 20 where a single patient 50 can stand is approximately 1.5 meters long. It is designed to accommodate the maximum step length of 2 to 3 steps for this patient 50, allowing convenient placement within a “personal” hydrotherapy pool.

The focus of this design lies in capturing the variations in “footprints” instead of evaluating the gait changes typically observed when patients walk on longer test walkways. This approach enables the evaluation and analysis of gait disorders in patients 50 and facilitates the implementation of a static pressure hydrotherapy exercise plan.

In FIG. 1A, the “adjustable” action of the adjustable pillars 10 is to raise the black walkway 20 to the ground surface or lower it underwater, facilitating the evaluation and exercise in different states, either on the ground or underwater.

Why is there a distinction between “ground surface” and “underwater”?

This is because buoyancy in water can assist the patient 50 in allowing greater movement. The patient 50 can practice walking with more normal or exaggerated gaits and gradually expand the range of motion. At the same time, the buoyancy presents challenges to balance and coordination. The patient 50's movements will be more effortless and relaxed compared to performing the same movements on land. Clearly, the footprints on the “ground surface” and “underwater” will exhibit different characteristic variations.

The “raising or lowering” action can be achieved using hydraulic or pneumatic systems or electric mechanisms driven by motors.

In FIG. 1A, there are two or more adjustable pillars 10 positioned at the corners surrounding the black walkway 20. They provide support, balance, and drive the vertical movement of the black walkway 20. The adjustable pillars 10 mainly consist of the sleeve columns 11 and the telescopic columns 12.

When the adjustable pillars 10 ascend, the telescopic columns 12 gradually retract into the sleeve columns 11, and when the adjustable pillars 10 descend, the telescopic columns 12 slowly extend out from the sleeve columns 11, as shown in FIG. 1B and FIG. 1C.

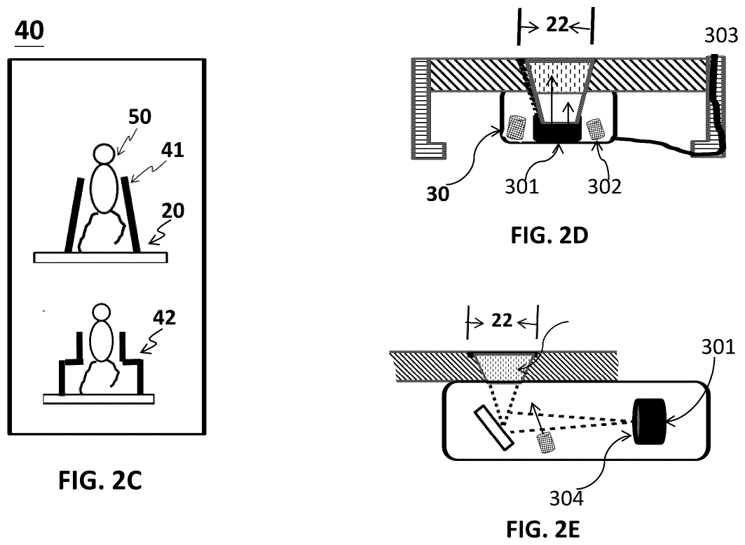

The adjustable pillars 10 further include a waterproof transmission cable (indicated as 303 in FIG. 2A) wrapped around them. This cable is used to transmit the multispectral images of the patient 50's gait captured by the multispectral thermal camera 301. The transmitted images are sent to a display monitor (not shown in the drawing) for monitoring, analysis, and recording by physical therapists or attending physicians.

At the upper end of the black walkway 20, there is a wide-based handrail 40 that provides support for the patient 50. At the lower end of the black walkway 20, there is a waterproof monitoring box 30. Inside this monitoring box 30, a multispectral thermal camera 301 (as shown in FIG. 2B) is installed, capable of capturing the gait images of the patient 50 by penetrating through the black walkway 20.

The reason for forming the black color on the black walkway 20 is mainly to “prevent the patient 50 from being distracted by looking down into the monitoring box 30 while standing on the black walkway 20 during the test.”

In addition to preventing the patient 50 from getting distracted, the black walkway 20 can also serve as a “distinction” from the transparent walkway boards used in previous technologies. This distinction allows the cameras mounted below the transparent walkway boards to capture the movements of individuals walking on the “transparent” walkway boards (facilitating visible light camera imaging).

In FIG. 1A, the wide-based handrail 40 is provided for the patient 50 to support their body and hold on with both hands. It is located on both sides of the black walkway 20. The wide-based handrails 40 on the left and right sides have an “unequal width” vertically. For example, the distance D2 at the upper end is smaller than the distance D1 at the lower end. This design prevents the patient 50's lower limbs from accidentally kicking the lower end of the wide-based handrail 40 when entering the black walkway 20.

In FIG. 1A, if a safer activity is desired on the black walkway 20, it is possible to use a device called a “body weight support harness” to partially suspend the patient 50's body, reducing the weight borne by the lower limbs during walking and enhancing walking ability. This suspension can be achieved by connecting suspension cables to a dedicated track on the ceiling.

Therefore, both the wide-based handrail 40 and the body weight support harness can be collectively referred to as the safety unit in this embodiment.

In FIG. 1A, if the patient 50 standing on the black walkway 20 for testing purposes does not have the safety unit, they can also use an assistive device such as a walker for support and balance while walking, and then remove the walker once they are positioned on the black walkway 20. This provides convenience for the patient 50.

In FIG. 1B and FIG. 1C, the dashed rectangular symbol labeled “protective” represents the adjustable black walkway platform 100. It indicates that the black walkway 20 can be positioned above ground level 201 (interpreted as an upper level) or below ground level 201 (interpreted as a lower level, corresponding to the concept of upper level). The lower level represents a personal hydrotherapy pool 60 utilizing static pressure.

In FIG. 1B, the diagram illustrates the principles and mechanism of evaluation and analysis on the ground surface. The adjustable black walkway platform 100 is typically positioned on the ground surface 201, allowing the entering patient 50 to directly “take position” on the black walkway 20 and wait for the testing to begin.

In FIG. 1B, if the adjustable black walkway platform 100 is situated on an actual “upper level” as found in everyday life, the patient 50 may first take an elevator to reach the upper level and then proceed to the black walkway 20 either independently or with the assistance of a walker.

In FIG. 1B, at the “lower level,” there is a static pressure hydrotherapy pool 60. By utilizing the raising and lowering action of the adjustable pillars 10, the black walkway 20 can be positioned on the ground surface 201 (upper level) or lowered into the pool water (warm water) 61 of the static pressure hydrotherapy pool 60 located on the ground surface 201 (lower level).

In FIG. 1B, when the black walkway 20 is positioned on the upper level, the patient 50 on the black walkway 20 can undergo evaluation and analysis according to the defined mode, which is referred to as the on-ground evaluation and analysis method 200.

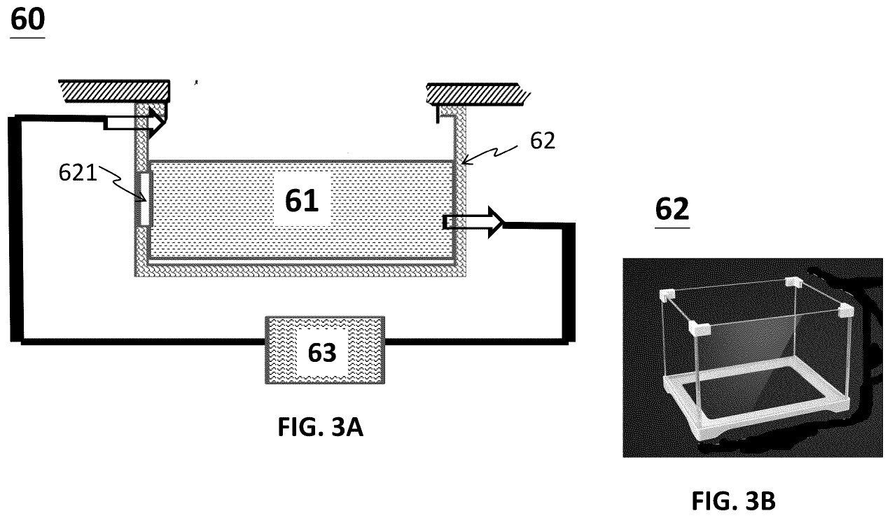

In FIG. 1B, the static pressure hydrotherapy pool 60 comprises pool water 61, the pool structure 62, a window 621, and water source device 63, which will be further detailed in FIG. 3A.

In FIG. 1C, the black walkway platform 20 is lowered into the pool water 61 of the static pressure hydrotherapy pool 60 through the raising and lowering action of the adjustable pillars 10. This immersion ensures that at least the lower half of the patient 50's body below the appropriate body position is submerged in the pool water 61.

The term “appropriate body position” primarily depends on the patient 50's height or the specific requirements of the test conditions (such as recommendations from physical therapists for patients with cardiovascular diseases), as illustrated in FIG. 3A of the static pressure hydrotherapy pool 60.

In FIG. 1C, when the black walkway 20 is positioned in the pool water 61, the lower half of the patient 50's body is immersed in the pool water 61 to receive evaluation and analysis. This mode is defined as the underwater evaluation and analysis method 300, which can also be defined as a type of static pressure hydrotherapy system.

In other words, a static pressure hydrotherapy system comprises a device that includes an adjustable black walkway platform 100 and a static pressure hydrotherapy pool 60.

In FIGS. 1, 1A, and 1B, the multispectral camera 301 captures multispectral images both inside and outside the static pressure hydrotherapy pool 60, utilizing the raising and lowering function of the adjustable black walkway platform 100.

These captured multispectral gait images of the patient 50, evaluated and analyzed on the ground surface and underwater, can be displayed on a monitor (not shown in the diagram). Physical therapists or attending physicians can observe and analyze these multispectral gait images for comparative evaluation and interpretation, either on-site or from recorded videos.

This comparative evaluation, which includes analyzing the differences between gait images on the ground surface and underwater, can be further extended to compare the multispectral gait images of normal individuals and different levels (mild/moderate/severe) of patients on the ground surface and underwater.

Regarding the evaluation of the patient 50's lower limbs (including gait/footprints) using multispectral thermal imaging, it is important to note that while the camera can evaluate the near-infrared and visible light images of the patient 50's lower limbs, the multispectral thermal camera 301 itself cannot penetrate the black walkway 20 and pool water 61. Therefore, it cannot provide a direct evaluation of the patient 50's lower limbs (including gait/footprints) using far-infrared thermal imaging.

In FIG. 1D, a VC (Vapor Chamber) temperature-controlled plate 80 is positioned at an appropriate distance from the patient 50, aligned with the multispectral thermal camera 301 and located beside the wide-based handrail 40.

The VC temperature-controlled plate 80 used in this embodiment is an improved version of a commonly used VC isothermal plate. It allows for temperature diffusion across the entire metal plate surface from a small point on the plate within a moment (approximately 3 seconds). In contrast, if the same thickness of copper metal plate (approximately 2 mm) were used without the VC technology, it would take several minutes to achieve temperature diffusion across the entire copper metal plate.

In this embodiment, the VC temperature-controlled plate 80 is designed with a semiconductor cooling element called a thermoelectric cooler (TEC) attached to the first side of the plate. The TEC functions as a point cold source, generating an instant cold temperature of approximately 0° C. The second side of the VC temperature-controlled plate 80 faces the lower limbs of the patient 50, allowing the multispectral thermal camera 301 to capture the thermal image of the patient 50's lower limbs from the side.

In simple terms, the purpose of the VC temperature-controlled plate 80 is to create different temperatures that serve as the “background” temperature for the multispectral thermal camera 301 when capturing the thermal image of the patient 50's lower limbs. This temperature difference between the “background” temperature and the temperature of the patient 50's lower limbs allows for a distinct and easily recognizable “appropriate temperature difference” by the thermal camera.

In other words, assuming T1 represents the generated “background” temperature and T2 represents the measured temperature of the patient 50's lower limbs, the mathematical equation for the desired temperature difference (ΔT) is expressed as T1−T2=ΔT. A larger value for ΔT makes it easier to distinguish the temperature difference.

As T2 is typically a constant value and T1 is a variable, providing a controller (not shown in the diagram) to control and generate an appropriate power supply to the VC temperature-controlled plate 80 allows for the determination of the “appropriate temperature difference.”

This “appropriate temperature difference” enables the multispectral thermal camera 301 to capture a clearer far-infrared thermal image 810 of the patient 50's lower limbs. This thermal image 810 can reveal any abnormalities before and after the evaluation and evaluation of the patient's lower limbs. The question is: How can we distinguish the thermal image of the patient's lower limbs?

During gait evaluation, it is important to have a view of the patient's lower limbs, at least below the knee joint, for a more accurate observation and evaluation of the overall gait using far-infrared thermal imaging. This allows for the observation of changes in the lower legs, footprints, size, patterns, step length, and step frequency during the gait process.

However, in FIG. 1D, when the patient 50 is in the pool water 61 as shown in FIG. 1C, it becomes difficult to distinguish the thermal image of the patient's lower limbs. It is challenging to discern the thermal image of the patient's lower limbs in FIG. 1C.

Based on the principles of most thermal cameras, they capture the “temperature (infrared radiation)” emitted from the surface of an object. Therefore, we can measure the temperature of the “gait/footprints” of the patient 50 as shown in FIGS. 1A and 1B. However, the measured temperature by the multispectral thermal camera 301 is not the true temperature of the “gait/footprints” because there is a certain degree of loss due to conduction through the black walkway 20, which absorbs a portion of the thermal radiation.

To obtain a clearer far-infrared “thermal image” of the patient's gait/footprints as shown in FIGS. 1A and 1B, there are two approaches: (1) reducing the thickness of the black walkway 20 to decrease the amount of heat absorbed by the walkway and (2) using a higher-resolution multispectral thermal camera 301.

In one or more embodiments of the present invention, the “thermal images” captured do not represent the actual temperature of the patient's gait/footprints or lower limbs. What matters is that these “thermal images” serve as a reference for the physiotherapist to compare and evaluate the differences between the “evaluation and analysis results” and the “results before and after hydrotherapy exercises.” The precise temperature values are not required for this purpose.

The human body's surface exhibits complex isotherms, with a wide range of temperatures that vary due to internal and external factors. The use of a multispectral thermal camera 301 allows for the evaluation of temperature in the patient's lower limbs or gait/footprints. Thermal imaging technology can provide insights into the biodynamic processes occurring within the human body. Temperature changes are often the first indicators of pathological processes in body tissues, even before functional or structural changes become apparent.

Therefore, using thermal images generated by a multispectral thermal camera 301 to evaluate gait disorders in patients like 50 and to detect potential pathological thermal changes can have significant diagnostic value in medical, health sciences, rehabilitation, physical therapy, and sports fields.

At least, the major advantage of a thermal imaging device lies in its non-invasive, non-contact nature, making it a safe tool for evaluation and analysis in research. In other embodiments of this invention, it is recommended to use thermal imaging devices of higher resolution models for observation.

The performance of a thermal imaging device is also influenced by the Instantaneous Field of View (IFOV), which refers to the angle covered by a single pixel of the detector. However, due to equipment errors, even with similar spatial resolution or IFOV, variations can be expected between different cameras.

Therefore, the “thermal images” in this embodiment serve only as references for comparative analysis during evaluation. However, among the various commercially available models of thermal imaging devices, no similar multi-spectral thermal imaging device as described in this invention has been found, capable of simultaneously capturing multi-spectral images including far-infrared (FIR), near-infrared (NIR), and visible light (VIS) for assessing gait disorders in patient 50.

The multi-spectral thermal imaging device 301, in contrast to a conventional thermal imaging device, includes two lenses: a first lens and a second lens. The first lens functions similarly to the disclosed first lens in a conventional thermal imaging device, capturing the far-infrared (FIR) wavelengths in the range of 8-14 μm. As for the multi-spectral thermal imaging device 301, the second lens is responsible for capturing two wavelength bands: visible light (VIS) in the range of 0.4-0.8 μm and near-infrared (NIR) in the range of 0.8-1.0 μm. However, the image sensor within the second lens of a conventional thermal imaging device restricts the capture of near-infrared (NIR) images within the range of 0.8-1.0 μm.

To capture near-infrared images with the second lens of the multi-spectral thermal imaging device 301, it typically requires an additional “near-infrared auxiliary light source” to illuminate the target and reflect back to the image sensor within the second lens, thereby enabling enhanced near-infrared imaging.

In some other embodiments, it has been observed that even without the projection of near-infrared light from the “near-infrared auxiliary light source,” the second lens can occasionally capture clear near-infrared images of the target object. Why does this happen?

The reason behind this may be the presence of natural near-infrared in indoor environments during daylight. However, during nighttime, it is not possible to have natural near-infrared as in daylight.

After the evaluation and analysis of patient 50 as described above, a subsequent “correlated and corresponding” exercise plan is developed based on the evaluated results for the same patient.

In this context, “correlated and corresponding” refers to the exercise steps or methods in the hydrotherapy regimen, which are planned or arranged by the patient's physical therapist or attending physician according to the evaluated analysis of the patient's performance “on the ground and underwater.” The exercise plan is tailored specifically to the individual patient's needs.

The exercise plan mentioned here implies that the underwater evaluation and analysis method 300 can serve both as an evaluation tool and an exercise plan.

One embodiment of the present invention involves capturing freeze of gait (FOG) images in patients with Parkinson's disease, processing them with different levels of transparency using the Alpha blending (o) technique, thus forming a multi-spectral thermal image for assessing FOG. Furthermore, this can provide physical therapists with a device and guidance for developing an “aquatic exercise plan” for patients with Parkinson's disease or elderly individuals.

The method of processing the multi-spectral thermal image for assessing freeze of gait (FOG) with different levels of transparency using the Alpha blending (σ) technique is similar to the method disclosed in patent application number 1757022. The only difference is the addition of a window image in the visible light portion, which will not be further elaborated here.

The evaluation and analysis include multi-spectral images of FOG in Parkinson's patients both “on the ground” and “underwater,” comparing the two conditions. The analysis includes parameters related to the temporal and spatial aspects of FOG.

The temporal parameters of gait include the rate of forward movement distance, the time elapsed between the first contact of one foot and the first contact of the contralateral foot, and the time elapsed between the first contact of one foot and the second contact of the same foot during a gait cycle. It also includes the observation of swing phase, which is the period when the foot is off the ground during a gait cycle, and other related parameters.

The spatial parameters of gait include the distance between the heels of both feet in the forward direction when the heel touches the ground during walking, among others.

In another embodiment of the present invention, the evaluation and analysis involve the patient (patient 50) performing gait exercises on the black platform (black step) both “on the ground” and “underwater.” Additionally, an “external stimulus” is applied to enhance the patient's focus during the exercise. This stimulus may increase the control of the patient's gait through the coordination area of the cerebellum. The goal is to observe the effectiveness of long-term implementation of gait movements in conjunction with this “external stimulus” and whether it can increase opportunities for limb activity while preventing falls.

In the evaluation and analysis involving patient 50 on the “ground” and “underwater” on the black step, it is assessed whether the effects before and after the patient's medication (levodopa) are related to the exercise results in the “underwater” condition.

Another embodiment of the present invention, tailored specifically for patient 50, includes setting reasonable goals for exercise on the black step in the “underwater” condition. The patient's achievement rate is regularly tracked, and periodic hospital visits are scheduled to assess the patient's performance during exercise in the “underwater” condition or on the “ground.” This helps identify when, where, and under what circumstances falls are most likely to occur in daily life, and appropriate exercises are provided to enhance the patient's functional independence.

It is worth noting that accurately capturing reliable information about gait characteristics at specific times, such as monitoring and assessing on the “ground” and “underwater” over time, can contribute to early diagnosis of the disease and its complications in patient 50, as well as finding the optimal exercise methods.

From a clinical perspective, the analysis of human gait disorders includes studies on gait characteristics in conditions such as multiple sclerosis, muscular atrophy, Parkinson's disease, bone marrow diseases, brain tumors and trauma, muscle spasms, senile dementia, heart disease, and physiological aging. While the described embodiments of the invention focus on the evaluation and analysis of gait disorders in patients with Parkinson's disease and freeze of gait (PD+FOG), it does not exclude the evaluation and analysis of gait characteristics in other conditions.

From a non-clinical perspective in the field of gait exercise and training, the research in the disclosed embodiments of the present invention focuses on designing balance training and exercise programs for patients with Parkinson's disease, including the use of static pressure hydrotherapy as an adjunct therapy to improve balance, prevent falls, and enhance cognitive function. This approach offers a more cost-effective solution compared to traditional evaluations and treatments, thereby maximizing the quality of life for patients with Parkinson's disease.

In clinical (long-term care, hospital) and non-clinical settings (nursing homes, independent living, etc.), discrete clinical evaluations during consultation periods often fail to detect changes in daily gait, despite changes in cognition, functional ability, or health status that may impact gait parameters until serious problems arise.

The gradual changes in gait parameters over time are not easily observed in daily life or brief outpatient visits. However, through the monitoring (including video recording) and evaluation of gait on the “ground” and “underwater” using multispectral imaging, as described in this invention, it becomes relatively easier to detect such gradual changes in gait parameters in a timely manner.

For the aforementioned multispectral imaging, further in-depth evaluation and analysis can include a method called “σ multispectral imaging” for sub-series interpretation. In this method, multiple images captured by the multispectral thermal imager 301 undergo a transparency blending process, forming an overlaid multispectral image that is easier for researchers to interpret. This is referred to as “σ multispectral imaging” in this invention, where “σ” represents “alpha blending”.

One or more embodiments of the present invention clearly adopt “multispectral imaging” as the method for evaluation, analysis, and tracking of training progress. This is primarily done to differentiate from prior art that discloses techniques involving multiple sensors worn by patients or capturing visible light images on transparent walkways.

Using a “wearable” technology solution with multiple sensors on patients poses a significant challenge, especially for elderly patients. Additionally, capturing images on transparent walkways can easily lead to distractions and loss of accuracy for patients like Patient 50.

Clearly, the aforementioned evaluation and analysis, along with the static pressure hydrotherapy training method, achieve the early diagnosis of gait disorders in patients by incorporating thermal imaging, visible light, and near-infrared gait footprint images before and after medication administration. This helps in finding the optimal exercise methods and their effectiveness.

The purpose of this invention is to detect and intervene early to assist high-risk populations in reducing the risk of falls, thereby minimizing the subsequent negative impact on the lives of high-risk elderly individuals and caregivers, reducing the physical, mental, and economic burdens of long-term care, and most importantly, improving the health and quality of life for patients with Parkinson's disease, Alzheimer's disease, other severe illnesses, and the elderly.

Another objective of this invention is to provide a static pressure hydrotherapy device that generates dual benefits of evaluation and exercise. It clearly distinguishes itself from conventional water therapy (pool) devices that only provide exercise functionality, thereby offering a more comprehensive rehabilitation solution.

Another objective of this invention is to provide a method for assessing and training gait-impaired individuals. The evaluation method includes the surface-based evaluation and analysis method (Method 200) as well as the underwater evaluation and analysis method (Method 300). The training method involves audio-visual stimulation combined with static pressure hydrotherapy.

Furthermore, this invention aims to replace the challenging “wearable” technology approach involving multiple sensors on patients, particularly for elderly patients, with a method that serves the purpose of evaluation, analysis, and training.

To achieve the basic principles and mechanisms of the present invention, namely the “multispectral imaging evaluation method for gait-impaired individuals” and the “static pressure hydrotherapy exercise device”, the adjustable black walkway platform 100 is raised above the indoor ground surface 201, and the platform is lowered into the static pressure hydrotherapy pool 60, both used for evaluation and analysis as well as static hydrotherapy exercise programs.

### Embodiment 1: Formation of a VC Temperature-Changing Plate 80

As shown in FIG. 1D, this embodiment improves the existing mature VC isothermal plate by attaching half of the first surface with a thermoelectric cooler (TEC) and a thermal insulation unit. Around the cooling point of the TEC, a thermal insulation unit (such as thermal insulation cloth or coating) is attached to prevent the heat generated by the TEC from dissipating on the first surface of the VC temperature-changing plate 80. Instead, the heat generated by the TEC is reflected entirely to the corresponding second surface, resulting in a more appropriate temperature difference for the lower limbs of the patient.

Therefore, this improved version of the VC isothermal plate is referred to as the VC temperature-changing plate 80.

### Embodiment 2: Formation of an Adjustable Walkway Platform 100

Refer to FIG. 2A as a schematic diagram of the black walkway, FIG. 2B as another schematic diagram of the black walkway, FIG. 2C as a schematic diagram of the wide-base handrail, FIG. 2D as a schematic diagram of footprint photography, and FIG. 2E as another schematic diagram of footprint photography.

FIGS. 2A and 2B provide further explanation of the black walkway 20 (non-transparent) as mentioned in FIG. 1A.

In FIG. 2A, the black walkway 20 in the embodiments of the present invention is made of black plastic (or black glass) material. It can also be formed using a composite matrix material, such as a combination of sprayed black aluminum alloy and bonded black plastic (or black glass).

Why does the black walkway 20 need to be black in color? When the patient with gait disorders (patient 50) is walking on the black walkway 20, if the material of the walkway is transparent, the patient might become “distracted” and curious to see what is underneath the walkway. This could interfere with gait evaluation and analysis.

However, if the material of the black walkway 20 is opaque black, how can the multispectral thermal imaging device 301 underneath the walkway capture the gait images of patient 50 for evaluation, tracking, and analysis?

There are various methods to form black plastic. For example, the inventor has disclosed related methods for producing opaque black materials in Taiwan Patent TWI423676 titled “Coating Substrate for Imaging Monitoring”, Taiwan Patent TWI328593 titled “Method and Application for Producing Infrared-Penetrable Black Plastic”, and the aforementioned Taiwan Patent TWI666935 titled “Microthermal Imaging Device for Enhanced Near-Infrared Image Capture”, which reveal the manufacturing methods for the opaque black “walkway” material and its ability to penetrate most near-infrared and a small portion of visible light. The detailed principles and mechanisms are not further described here.

In other embodiments of the present invention, a simple method to produce black plastic is to mix a black colorant called carbon black into transparent plastic materials (such as PMMA or PC).

Sometimes, to “reduce” the weight of the black walkway 20, it can be made of a composite material of aluminum alloy and black plastic.

In terms of weight, aluminum alloy is lighter than black plastic, and black acrylic is lighter than black glass for the same volume.

In a preferred embodiment of the present invention, black plastic is used because it is easier to process (drilling and assembly), such as in FIG. 1A, where it is easier to set up the lifting column 10 and wide-base handrail 40 on the black walkway 20.

In FIG. 2A, a testing area 22 is set in the center of the front side of the black walkway 20, and the range of gait activities of all tested patients 50 is “recommended to be limited” within this testing area 22.

How can the patient 50 be “recommended to be limited” within this testing area 22? (1) It can be observed directly by a physical therapist or the patient 50 themselves through a liquid crystal display placed above the black walkway 20. (2) The area of the testing area 22 can be slightly raised (about 2-5 mm) compared to the flat surface of the black walkway 20, allowing the patient 50 to experience and perceive the boundary.

Because there is a monitoring box 30 installed underneath the testing area 22, and the camera (such as the multispectral thermal imaging device 301) inside the monitoring box 30 is aligned with the testing area 22 to capture the gait pattern of the patient 50 standing on the testing area 22.

In FIG. 2B, on the reverse side of the black walkway 20 corresponding to the position of the testing area 22 (indicated by dashed lines), a scale 23 is marked. The purpose of this scale 23 is to serve as a reference length for the gait of the patient 50 on the testing area 22. Both the testing area 22 and the scale 23 are printed with “non-transparent white paint” as white lines on both sides of the black walkway 20.

In FIGS. 2 and 2A, the “non-transparent white paint” cannot penetrate visible light and near-infrared. Therefore, the multispectral thermal imaging device 301 can “penetrate” the black walkway 20 and capture the images of the white lines on the testing area 22 and the scale 23, as well as the gait images of the patient 50 in near-infrared and visible light.

FIG. 2C further illustrates different patterns of the wide-base handrail 40 shown in FIG. 1A, such as the inclined shape 41 or the N-shaped 42. Its main function is to prevent the patient 50 from hitting the lower end of the wide-base handrail 40 when entering the black walkway 20, as shown in FIG. 1A with D1 (distance 1)>D2 (distance 2).

FIG. 2D shows the proper placement of the monitoring box 30 in the center underneath the black walkway 20. Inside the monitoring box 30, there is a multispectral thermal imaging device 301.

When the imaging lens of the multispectral thermal imaging device 301 is aligned with the gait of the patient 50 on the testing area 22 on the black walkway 20, in order to obtain a more complete image, the captured image range of the multispectral thermal imaging device 301 must cover the testing area 22.

To obtain a clearer image, additional “near-infrared auxiliary light source” 302 may be required. The near-infrared auxiliary light source 302 mainly consists of IR-LEDs (infrared light-emitting diodes) with a wavelength of 940 nm. It allows the multispectral thermal imaging device 301 to capture clearer near-infrared gait images. The reason for using IR-LEDs with a wavelength of 940 nm is that they do not emit visible red light dots (red dots) when in operation. Otherwise, if there were “red dots” displayed above the black walkway 20, it could distract the patient 50 and affect their focus.

The definition of “940 nm” mentioned above refers to “near-infrared that is not visible or discernible to the human eye,” which excludes the commonly used 850 nm IR-LEDs.

In this case, the near-infrared centered around “940 nm” is also defined as “940±20 nm” near-infrared. The range of “±20 nm” accounts for the reference error range of IR-LEDs manufactured by suppliers, and it is characterized by being “completely invisible to the human eye.” Other commonly available near-infrared particles with centered wavelengths of 960 nm, 980 nm, etc., should also be considered as part of the “940 nm-centered” family.

In FIG. 2D, the junction between the monitoring box 30 and the black walkway 20 must have a “waterproof” function to prevent water from the pool 61 from entering the monitoring box 30.

In FIG. 2D, the transmission cable 303 for transmitting the video signal from the multispectral thermal imaging device 301 inside the monitoring box 30 passes through the black walkway 20 and is led out. After being led out, the transmission cable 303 is transmitted through the adjustable support column 10 and then sent to a suitable position indoors to a display device (not shown in the FIG.) for viewing the gait images of the patient 50.

The transmission cable 303 can pass through the interior of the telescopic column 12 of the adjustable support column 10 and be led out from the top of the telescopic column 12 (near the sleeve column 11) by creating a hole. Even if the outer edge of the transmission cable 303 is waterproof or meets safety specifications, at least when the transmission cable 303 is led out from the top of the telescopic column 12, it will not come into contact with the pool water (warm water) 61.

The transmission cable 303 transmits the multispectral gait images of the patient 50 standing on the black walkway 20, allowing the patient's physical therapist or others nearby to observe or record the images.

In FIG. 2D, it is also possible to retrieve and play back the stored images from the SD card inside the multispectral thermal imaging device 301 after leaving the hydrostatic water therapy system 60. This eliminates the need for setting up the transmission cable 303.

In FIG. 2D, the power source for the near-infrared auxiliary light source 302 can be a rechargeable battery (not shown in the FIG.).

In FIG. 2D, since there may be a need to repair the multispectral thermal imaging device 301 and the light source 302 inside the monitoring box 30, it is necessary to use “gaskets” between the monitoring box 30 and the black walkway 20 to ensure waterproofing (not shown in the FIG.).

In situations where the captured image range of the multispectral thermal imaging device 301's camera in FIG. 2D is insufficient to cover the test area 22 due to inadequate depth of field, the focal length of the camera can be extended. In FIG. 2E, this can be achieved by using a reflector 304 to reflect the image into the test area 22 or by directly attaching a suitable magnifying lens to the camera to enlarge the image to cover the test area 22.

### Embodiment 3: Formation of a Hydrostatic Water Therapy System 60

Please refer to FIG. 3A for an illustration of the hydrostatic water therapy system and FIG. 3B for an illustration of the glass pool body.

In FIG. 3A, the hydrostatic water therapy system 60 consists of pool water 61, the pool body 62, and water source device 63.

The “static pressure” in the hydrostatic water therapy system 60 refers to the hydrostatic pressure. It is the pressure exhibited by the warm water 61 when it is at rest inside the pool body 62. Assuming that the only external force acting on the warm water 61 is gravity, the hydrostatic pressure at any point within the warm water 61 region is isotropic, meaning the pressure at the same point is the same in all directions.

Furthermore, in the hydrostatic water therapy system 60, apart from the natural “convection” of the warm water 61 caused by the movement of the patient 50 within the region, it is important to minimize external interference (such as from bystanders) that could indirectly affect the quality of the multispectral thermal images captured by the multispectral thermal imaging device 301.

For example, in the case of patients 50 with certain cardiovascular conditions, their physical therapists may recommend adjusting the height of the warm water 61 to be below their “chest” level to reduce water pressure. This can be achieved by controlling the height of the adjustable support pillar 10.

Clearly, the hydrostatic water therapy system 60 is designed as an individual pool for the patient 50, distinct from shared swimming pools. However, it is possible to set up multiple “individual pools” side by side in a spacious rehabilitation room.

The term “warm water” refers to water at a temperature suitable for the human body, typically adjustable between approximately 30 to 37° C. However, within the permissible range of training, it is not excluded to use temperature-controlled warm water, as it can provide a soothing and warm temperature for the patient 50, helping to relax muscles and alleviate stiffness and pain.

Since the water in the hydrostatic water therapy system 60 is usually kept warm, it is important to maintain a warm and comfortable air temperature to prevent tremors in patients with Parkinson's disease (PD) and provide a pleasant experience. Even patients with more advanced stages of PD can benefit from immersion in the hydrostatic water therapy system 60.

This is also the reason why the adjustable black platform 100 in the form of a lift in FIG. 1C must be capable of submerging to any desired depth within the water tank body 62. It is designed to accommodate the needs of patients 50 with different heights or those with various other medical conditions.

Clearly, the structure and features of the adjustable black platform 100 in this case, in conjunction with the system and method disclosed in Patent TWI757022 titled “System and Method for Gait Footprint Analysis Based on Alpha-Type Multispectral Imaging”, differ significantly in terms of their technical field and operational effectiveness.

As for the material of the water tank body 62, it can be constructed using common building materials such as cement, tiles, fiberglass, or reinforced glass, among others. However, around the water tank body 62, one or more transparent “windows” 621 can be incorporated to allow physical therapists, attending physicians, family members, and other visitors to observe the activities of patients 50 in the warm water 61. These windows can also serve as openings for external cameras to record the hydrotherapy process.

In FIG. 3B, in order to retain one or more “windows” in the water tank body 62, the case includes an alternative embodiment that utilizes reinforced, thick transparent glass (e.g., 9 mm thick) that has undergone strengthening treatment. The shape of these windows can be square or circular, depending on the design of the adjustable black platform 100. These windows form one or more “windows” 621.

The captured video footage of the hydrotherapy process through the “windows” 621 is referred to as “window images.” These window images, when compared with the multispectral images captured by the multispectral thermal imaging device 301 as shown in FIG. 2C, can provide further evaluation and analysis of the benefits of static pressure water therapy and exercise.

The water source device 63 includes functions such as circulating, replacing, cleaning, disinfecting, and controlling the temperature of the warm water 61. It also provides both cold and hot water supply.

### Embodiment 4: Basic Gait Image Recognition

To further understand the multispectral images captured by the multispectral thermal imaging device 301 in relation to basic gait patterns, especially as shown in FIG. 2C, where the device captures far-infrared, near-infrared, and visible light images of patients 50 performing various gait patterns on the black platform 20.

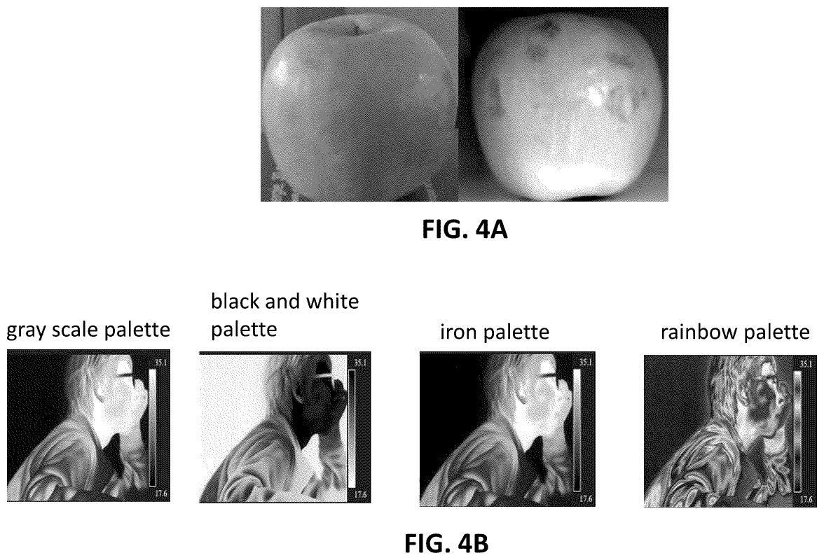

The thermal images of the gait patterns are depicted in FIG. 1D and FIG. 4B. The near-infrared images of the gait patterns are shown in FIG. 4A and FIGS. 8-9A. The visible light images of the gait patterns include visual observations and the window images depicted in FIG. 3A.

It is important to note that in one or more embodiments of this invention, the emphasis of evaluation and analysis is largely placed on the near-infrared images. However, this does not limit the analysis to solely near-infrared images as the only method in the present invention.

Please refer to FIG. 4A, which illustrates the features of near-infrared images, and FIG. 4B, which represents the color palette of the multispectral thermal imaging device.

In FIG. 4A, two images of the same apple are shown: the left image represents the visible light image, and the right image represents the near-infrared image. Both photos were taken shortly after purchasing the apple.

It is evident that the near-infrared image clearly reveals signs of surface damage that are not visible in the visible light image, demonstrating the unique characteristics of near-infrared (NIR) imaging.

In FIG. 4A, near-infrared imaging has unique advantages. Apart from its ability to penetrate deep tissues in the soles of the feet, its main advantage lies in its higher contrast compared to visible light when observing objects and their surrounding environment. Without good contrast, even in bright sunlight, visibility would be limited, as seen in the visible light image on the left.

However, darker objects can be challenging to detect in a dark background, resulting in poor contrast. This is why near-infrared auxiliary light source 302, as shown in FIG. 2C, is employed to compensate for the lack of visible light. The thermal images captured by the multispectral thermal imager 301 are independent of the presence of visible light, allowing for mutual evaluation and comparison.

As depicted in FIG. 4B, the multispectral thermal imager 301, similar to a conventional thermal imager, is equipped with a feature known as a “color palette.” This color palette, processed by image software, generates different false-color display indexes for the images captured by the multispectral thermal imager 301.

For instance, different color palettes such as the “gray scale palette” are particularly useful for resolving small geometric details but may not effectively display small temperature differences. The “iron palette” is highly intuitive and easily understood by individuals without thermal imaging experience. The “rainbow palette” offers vibrant color variations between light and dark, resulting in enhanced contrast. However, it may introduce noise in the image when dealing with surfaces or multiple temperatures.

In fact, different color palettes have different interpretation purposes, allowing physical therapists to discern subtle differences. Conventionally, the “black and white palette” assigns “black” to the coldest temperature in the thermal image and “white” to the hottest temperature, with varying shades of gray in between.

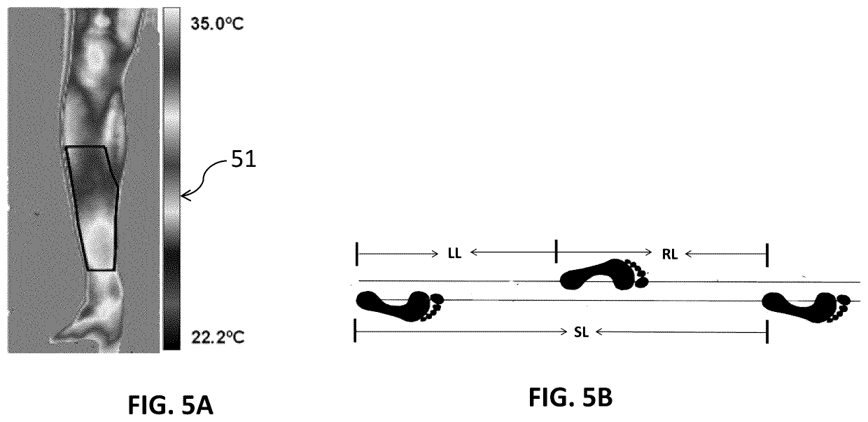

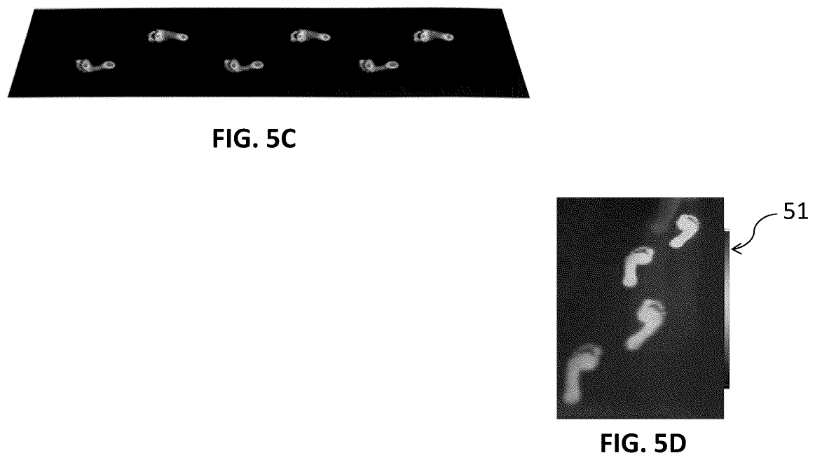

Please refer to FIG. 5A for an illustrative diagram of lower limb thermography. FIG. 5B depicts a schematic representation of a normal gait footprint, while FIG. 5C and FIG. 5D illustrate thermal images of gait footprints.

In FIG. 5A, an example is provided for capturing lower limb thermography as described in FIG. 1D, referencing the VC variable temperature plate 80. The light blue background in the image represents a significantly lower temperature than the lower limbs. On the right side of the image, a “pseudo-color temperature bar 51” indicates the temperature distribution of the lower limbs. The black rectangular box shown within the lower limbs may represent an area of interest for research by medical professionals.

FIG. 5B displays the pattern of a normal gait footprint for an individual, with “LL” representing the length of the left footstep, “RL” representing the length of the right footstep, and “SL” representing the stride length. Typically, LL and RL are equal in a normal gait.

FIG. 5C demonstrates an example of a thermal image capturing a footprint on the black pathway 20. Such a thermal image can only be captured clearly by a high-resolution thermal imager with a resolution of approximately 320×240 or higher.

It is worth noting that the gait footprint shown in FIG. 5C is a thermal image of the residual footprint left by patient 50 on the black pathway 20. This “thermal image” may disappear within approximately 1 to 5 seconds due to the “convection” of the surrounding air.

In FIG. 5D, another color palette of the thermal image of a footprint on the black pathway 20 is shown, with a “pseudo-color temperature bar 51” on the side indicating the temperature distribution of the footprint.

It is worth noting that from the thermal images shown in FIGS. 5, 5B, and 5C, clear thermal images can be captured even in a completely dark environment (such as during a sudden power outage), which is a characteristic feature in gait patterns.

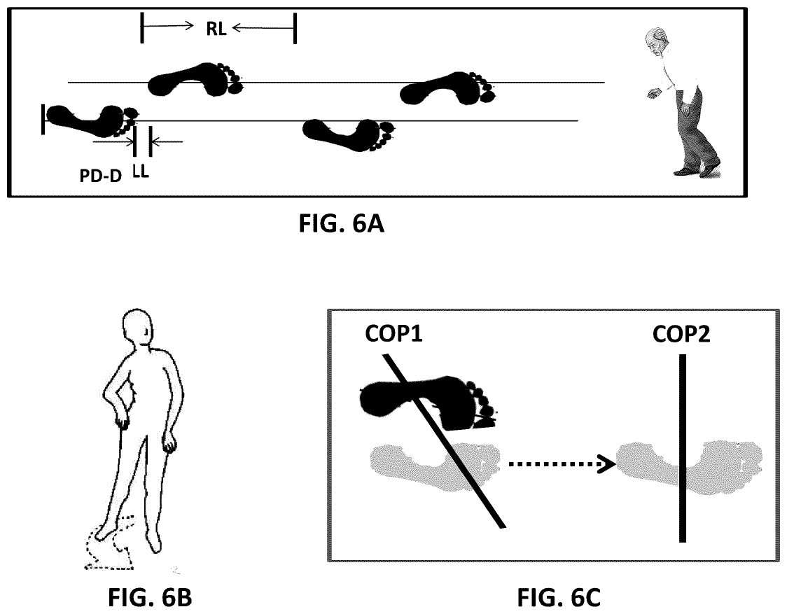

Please refer to FIG. 6A for an illustrative diagram of freezing of gait (FOG) in gait patterns. FIG. 6B depicts an illustration of freezing of gait, and FIG. 6C illustrates the center of pressure (COP) in Parkinson's disease (PD).

Freezing of gait (FOG) is a common symptom observed in patients (referred to as patient 50) with Parkinson's disease (PD). It is characterized by sudden interruptions and severe difficulty in walking, where the patient 50 feels as if their feet are “glued” to the ground, making forward movement impossible. In addition, as shown in FIG. 6B, freezing of gait is often observed during “turning” and when approaching a destination, as well as in narrow or obstructed spaces. Patients 50 with PD and FOG have an increased risk of falling and a decreased quality of life.

The clinical features of patients 50 with PD and FOG (PD+FOG) are as follows: (1) In episodes, the feet or toes cannot lift off the ground. (2) During episodes, both legs tremor alternately at a frequency of approximately 5 Hz. (3) Prior to episodes, there is often a shortening of stride length and an increase in walking speed. (4) Episodes can be alleviated by external stimuli. (5) Episodes exhibit asymmetry, primarily affecting one lower limb or occurring during turns. These symptoms significantly impact the patient's quality of life.

The mechanism underlying FOG in PD patients 50 is still not fully understood in the global medical community. Preliminary research findings from certain international medical literature suggest that FOG may be attributed to the dysfunction of multiple brain regions.

Therefore, this embodiment does not involve pathological medical research on the FOG symptoms experienced by patient 50. The present disclosure also does not purport to provide sufficient medical resources or equipment. In several embodiments, it only provides physical therapists with preliminary evaluation and analysis of patient 50 using multispectral imaging. The purpose is to assist in developing corresponding aquatic exercise programs that can be used as a reference for physical therapists and related personnel to improve the patient's quality of life.

In FIG. 6, a gait pattern of patient 50 with FOG is shown. In each step, a small distance FOG-D can be observed between the left and right feet. As depicted in the diagram, when the left foot takes a step forward, the right foot cannot “cross over” the left foot as a normal person would, and it consistently remains a distance FOG-D behind the left foot. This phenomenon resembles a propulsive gait or “shuffling gait.”

One of the objectives of the present invention is to explore whether this “shuffling gait” phenomenon in the hydrostatic water therapy system 60 is influenced by external forces or resistance, and further identify methods for exercise or treatment.

In FIG. 6C, a gait pattern of patient 50 with FOG characterized by “head down and forward-leaning posture” (COP1) is shown. This forces the right foot to take a step forward in an attempt to maintain balance (COP2).

The physical therapist in this embodiment can evaluate the movement of the center of pressure (COP) in patient 50's body displacement using visual observation and the three multispectral footprint images. This provides multiple sets of data for variations, enabling the identification of optimal guidance for exercise methods.

Now, the reasoning method for exploring the “linkage” between the center of pressure (COP) or gait balance and multispectral imaging in this embodiment will be explained.

Gait analysis is a fascinating and complex field. The ankle joint, which requires both stability and mobility, poses a seemingly conflicting challenge. This complexity makes the evaluation and analysis process quite intricate as it involves considering the overall biomechanics of the lower limbs rather than focusing solely on the footprints.

However, for now, let's “focus” on this specific area of “footprints” to facilitate understanding.

The center of pressure (COP) can be used to assess the balance ability (body control) of patient 50. Literature indicates that the center of pressure in maintaining a static posture reflects the position of the body's center of gravity. Therefore, it can represent the sway of the body's center of gravity, COP trajectory, maximum anterior/posterior and medial/lateral deviations, as well as the COP area. These are all indicators for evaluating balance ability.

In other embodiments, patient 50 can be instructed to perform the evaluation with their eyes open or closed, aiming to gather more COP trajectory image data. Through the analysis of this COP trajectory image data, it is possible to explore how to enhance patient 50's balance ability and predict the likelihood of falls in the future.

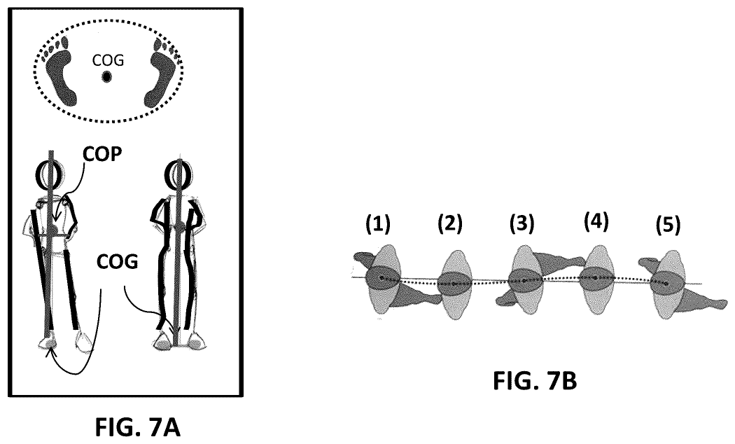

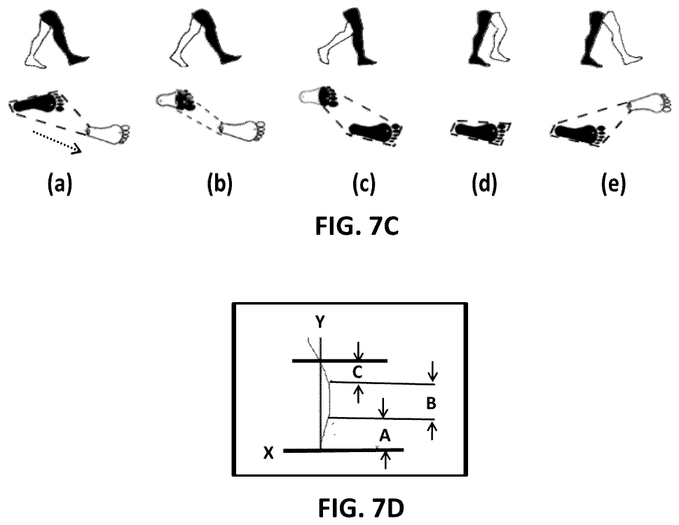

Please refer to FIG. 7A for schematic diagrams of the vertical line of the body's center of gravity. FIG. 7B, FIG. 7C, FIG. 7D, and FIG. 7D illustrate different representations of the vertical line of the body's center of gravity.

In FIG. 7A, when patient 50 is standing, the area enclosed by both feet (within the circular dashed line) is referred to as the base of support (BOS). In the center of this area, there is a point representing the center of gravity (COG) of patient 50. Maintaining balance is achieved when the center of pressure (COP) passes through this COG point. However, in the case of standing on one foot (with the center of support already shifted), a red line is drawn upward from the COG point. Balance can be maintained if the COP passes through this line; otherwise, there is a risk of falling.

In FIG. 7B, as patient 50 walks, a slight lateral displacement of the body's center of gravity (represented by the dashed line) can be observed from the central line of the head (solid line).

In FIG. 7C, when patient 50 walks on the black pathway 20, within the dashed boxes in (a) to (e), the black region indicates the movement of the body's center of gravity toward the white region. For example, in (a), the center of gravity is initially on the left foot, and as we progress to (b), it slowly moves towards the right foot. The reduction of the black region observed from the left foot in (b) indicates the movement of the body's center of gravity towards the white region. This cycle repeats until (e) is completed.

When drawing a vertical line through the center of support during single-leg stance, the absence of crossing with the COG prevents falling.

Typically, patient 50 should pay attention to three processes: (1) the COG point supporting body weight, (2) the center of support during single-leg stance, and (3) ensuring that footprints remain within the patient's base of support (BOS) during the swinging leg step. It is crucial to observe the differences in footprints during these three processes and conduct cross-references.

Therefore, based on the movement of the body's center of gravity towards the white region as described in FIG. 7C (a) to (e), it can be inferred that obtaining multispectral images of the black portions in (a) to (e), particularly high-contrast near-infrared images (such as FIGS. 4, 10A, 11B, 11D), and observing the differences in movement in these near-infrared images can be used to assess and infer the balance ability of patient 50.

In FIG. 7D, we present the correlation between the captured images and COP displacement trajectories based on the descriptions in FIGS. 7A and 7B, dividing the evaluation and analysis into three stages: A, B, and C.

For example, by examining the displacement from (1) to (2) in FIG. 7B and from (a) to (c) in FIG. 7C, we can observe an example of the corresponding COP displacement trajectory in region A of the Y-axis in FIG. 7D.

Based on this example of the related COP displacement trajectory, we can assess and analyze the body's “sway” of patient 50.

However, the extent of this “sway” differs between being on the ground and being underwater due to the influence of gravity. In other words, by evaluating the displacement generated on the ground and underwater and conducting repeated evaluation and analysis, we can determine the level of “benefit” obtained by patient 50 before and after hydrotherapy exercises in the hydrostatic water therapy system.

The “sway” velocity refers to the distance the COP moves per unit time and is related to the ability to control posture. It can be used to assess variations in patient 50's strategies for maintaining body posture stability and changes in muscle contraction and extension during hydrotherapy exercises.

The evaluation of patient 50's standing posture balance within the base of support (BOS) range depicted in FIG. 7A can be divided into two categories: static and dynamic. Static balance refers to patient 50 maintaining a fixed position without movement or rotation, while dynamic balance involves maintaining a certain trajectory or movement along the rotation axis within the testing area 22 without any external interference.

Inference of COP displacement trajectory parameters such as total length, area, anterior-posterior and medial-lateral range can be used to assess patient 50's balance ability. The multispectral images of the “footprints” are used to evaluate the position of the body's center of gravity, representing the sway of the body's center of gravity, COP displacement trajectory, maximum anterior-posterior and medial-lateral displacement, as well as COP area. All these parameters serve as indicators for assessing balance ability (ability to control the body).

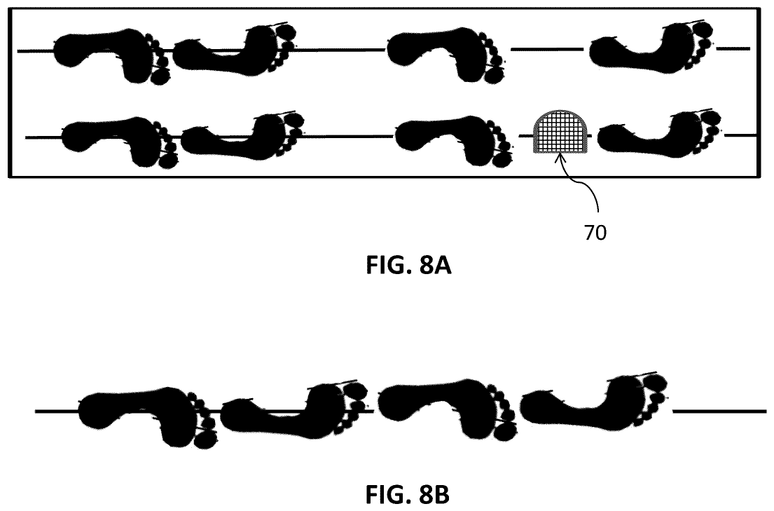

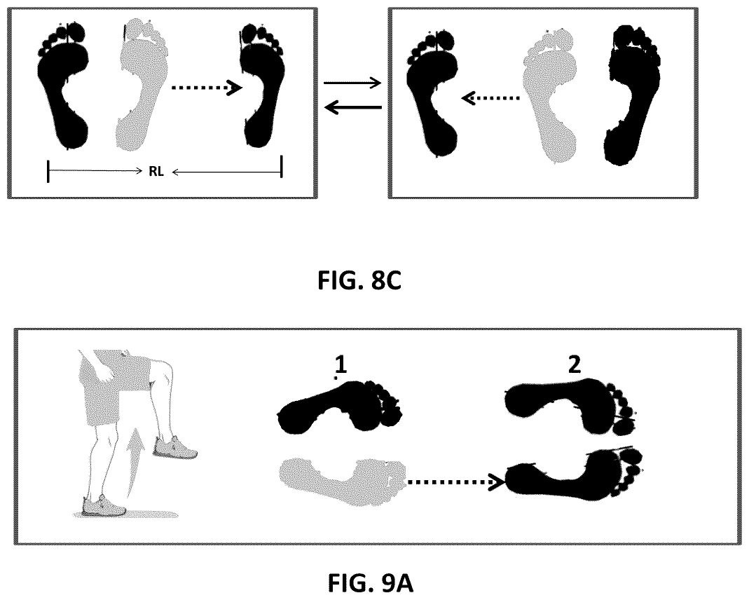

Please refer to FIG. 8A for illustrations of guided exercise. FIGS. 8B and 8B provide additional examples of guided exercise for patient 50 based on the physical therapist's evaluation and analysis.

For patients with mild to moderate symptoms, in addition to basic walking exercises, further challenging exercises can be performed on the ground using evaluation and analysis method 200 and underwater using evaluation and analysis method 300.

In FIG. 8A, during the initial stage, patient 50 takes a small step forward with both feet close to the ground, followed by a larger step with both feet. Then, on the next day or session, a soft obstacle 70 is added between the feet during the larger step, and patient 50 “steps over” the obstacle. For patients with moderate symptoms, this exercise on the ground may require assistance from others to help alleviate the fear of falling and maintain a positive attitude towards the exercise. However, in the underwater setting, patient 50 may feel more secure and confident in their balance during the exercise.

In FIG. 8B, patient 50 takes continuous small steps forward with both feet close to the ground, challenging their sense of balance.

In FIG. 8C, patient 50 stands with both feet firmly planted and then laterally extends the right foot to the right and brings it back, followed by a similar movement with the left foot to the left. This action is repeated several times.

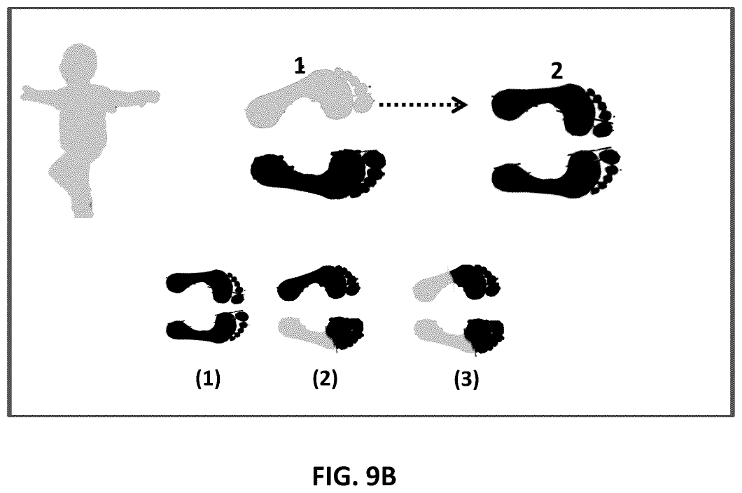

Please refer to FIG. 9A for illustrations of foot lifting exercises. FIG. 9B provides an additional illustration of this exercise.

FIGS. 9A and 9B show examples of foot lifting or single-leg exercises, where “1” indicates lifting the foot or performing the exercise (depicted by the gray FIG.), and “2” indicates returning to the starting position or standing on one foot or both feet, with the toes (black) touching the ground and the heels (gray) raised. This action is repeated several times.

Furthermore, based on FIGS. 7 to 7B, physical therapists can perform the evaluation and exercise described in FIGS. 8 to 9A using evaluation and analysis methods 200 and 300 on the ground and underwater. The imaging data of patient 50 can be further referenced and studied to improve the treatment approach.

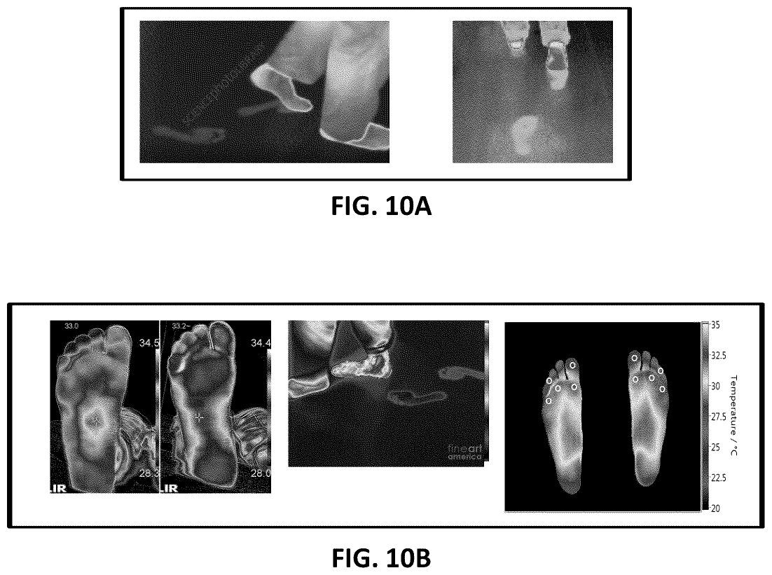

Please refer to FIG. 10A for an illustration of multispectral thermal imaging. FIG. 10B shows an example of multispectral near-infrared imaging.

As shown in FIG. 10A, it depicts a multi-spectral “thermal” image of a gait. Medical research often focuses on temperature analysis within gait images because temperature variations are observed before and after movement in gait-impaired individuals. For instance, thermal imaging can reveal distinct temperature differences in cases of falls or inflamed tissues.

It is worth noting that in the depicted “thermal” image in FIG. 10A, when displayed on the black walkway (20), the multi-spectral thermal imaging device (301) must capture images instantly through photography or video recording. Otherwise, these thermal images remaining on the black walkway (20) may disappear within a short period of time (e.g., 1 to 5 seconds) due to thermal dissipation caused by air convection near the black walkway.

FIG. 10B represents a processed multi-spectral image of a gait using a color palette (as shown in FIG. 4B). In several embodiments of this application, for the evaluation and analysis of patient 50 using on-road analysis method 200 and underwater analysis method 300, professionals such as medical practitioners and physical therapists commonly collaborate to assess these relatively complex color palette images.

Embodiment 5 presents an evaluation and analysis system for assessing the risk of abnormal gait using multi-spectral imaging. The system includes the following components: (1) Evaluation modes, (2) Evaluation methods, (3) Evaluation subjects, (4) Evaluation items and images, (5) Visual and auditory guidance methods, and (6) Physical therapist coaching methods.
(1). Evaluation Modes: The system comprises two evaluation methods: on-road analysis method 200 and underwater analysis method 300.
   (1-1): On-road analysis method 200, as shown in FIG. 1B, involves assessing the patient's activities while standing on the black walkway (20) placed on the floor.
   (1-2): Underwater analysis method 300, as shown in FIG. 1C, involves submerging the lower body of the patient in the pool (61) while standing on the lowered black walkway (20) and evaluating their activities.
(2). Evaluation Methods:
   (2-1): The system allows for analysis with the patient's eyes open or closed. Multiple analyses can be conducted using the chosen evaluation mode (1) to gather more deviation data regarding the patient's Center of Pressure (COP) movement trajectory images.
   (2-2): Utilizing the COP movement trajectory image data, the system aims to enhance the patient's balance capabilities and predict their risk of falling.
(3). Evaluation Subjects:
   (3-1): Patients with high dependency or requiring assistance in walking.
   (3-2): Patients with mild to moderate impairment who can walk independently without assistance.
   (3-3): Normal individuals serving as a control group.
(4). Evaluation Items and Images:
   (4-1): Evaluation includes the distribution of “thermal” images of the contact area between the patient's feet and the ground and the lower limb.
   (4-2): Evaluation includes “visible light” and “near-infrared” images of the patient's footprints' size, gait speed, stride length, step frequency, and Center of Pressure (COP) movement.
(5). Visual and Auditory Guidance Methods:
   (5A): Visual guidance group.
   (5B): Auditory guidance group.

The purpose of the visual and auditory guidance methods is to determine: (5-1) Whether there is a significant effect on gait training when providing external “cues” to patient 50. (5-2) To conduct comprehensive visual and auditory guidance tests, including patient 50 as the control group and motivated participants in therapy as the reference group.

To facilitate the gait evaluation and training effectiveness for patient 50, it is important to emphasize that the exercises should be performed consistently and with high repetition under the influence of medication. Variations should be minimized. Additionally, clear and straightforward visual and auditory guidance should be provided to assist patient 50 in executing the movements.

Furthermore, it is recommended to encourage patient 50 to elongate their “stride” as much as possible during the visual and auditory guidance. The training content should be adjusted according to the individual's capabilities to help build confidence.
(5A1) Visual guidance group: Patient 50 observes a monitor (or television) screen that displays their position within the testing area (22) of the black walkway (20). A “•” mark is displayed if patient 50's steps align correctly with the indicated instructions, while an “x” mark is displayed along with instructions to adjust forward, backward, right, or left if the steps do not align correctly.
(5B1) Auditory guidance group: Patient 50 listens to a sound, such as a “thump,” and takes a “step forward” upon hearing it. Patient 50 repeats this process for each subsequent “thump” sound. The time interval for each “thump” sound (N) can be set between 0.5 to 2 seconds. Assuming there are X different segments (e.g., N=0.5, 1 . . . 2), these segments can be adjusted based on patient 50's physique, age, or gender.
(6) Physical Therapist Coaching Method:
(6-1) Since multiple evaluations may be necessary, it is important to allow patient 50 appropriate rest intervals to prevent gait changes due to excessive fatigue or pain.
(6-2) If patient 50 experiences sudden freezing or cramping of the feet during training, where it feels as though the feet are stuck to the pool floor, patient 50 should be instructed to try “lifting the toes.” This action may help restore the mobility of the feet. Otherwise, the movement should be stopped. Patient 50 can take a break and perform deep breathing exercises 2-3 times to relax and calm down.
(6-2-1) In the event of an emergency situation with patient 50 during the guidance session, where immediate handling becomes difficult, it is necessary to promptly refer patient 50 to specialized emergency care.
(6-3) Patient 50 should be mindful of the concept that the static water pressure and water depth are directly proportional. For example, the higher the water level, the greater the burden on patient 50's heart.
(6-3-1) The water depth can be adjusted based on patient 50's comfort level. It can be gradually increased or maintained at the same depth. The exercise, as shown in FIGS. 7 to 8A, is typically performed for about 10-15 minutes.
(6-4) Patient 50 should sway the body's center of gravity gently from side to side or back and forth in rhythm with the music of the auditory guidance method (5). This prepares patient 50 to take the first step.
(6-4-1) Patient 50 should be ready to take a big step. The Center of Pressure (COP) is shifted to one foot, and the other foot is extended with a large step. Patient 50 takes big strides following the rhythm of the auditory guidance method (5), stepping out with the first step after releasing from the freezing of gait (FOG).

The effectiveness of these methods depends on individual patient 50 and the background for determining which approach to apply. From a physiological perspective, this is also a self-regulation mechanism for patient 50.

The parameter space involved in the multi-spectrum image analysis of gait footprints reflects the “relative percentage of time within the entire gait cycle specific to patient 50”.
(6-5) Literature data shows that patients 50 often spontaneously create new gait patterns to overcome their walking difficulties and maintain the movement of the Center of Pressure (COP) and independence. In such cases, the physical therapist should teach patient 50 about different educational aspects to help them understand and find methods that are more suitable for their unique situations.

Additionally, it can be beneficial to incorporate the auditory guidance method (5) or utilize a specially designed projection-based static treadmill for cycling to help patient 50 develop new balance techniques.

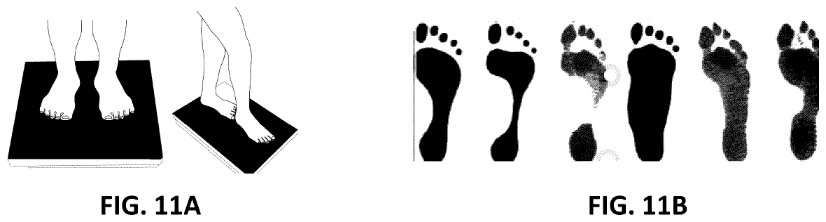

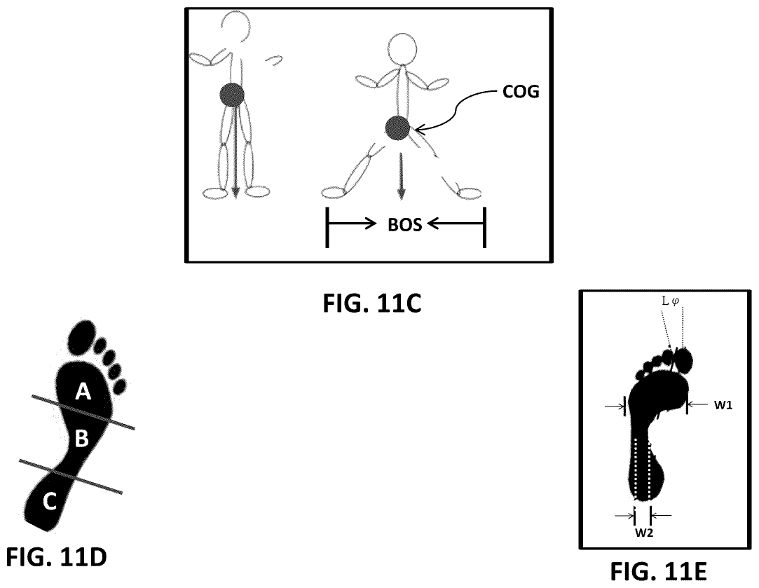

Please refer to FIG. 11A for the schematic illustration of standing on the black step pathway. FIG. 11B represents various patterns of footprints, FIG. 11C demonstrates the stability of standing, FIG. 11D shows the image area of footprints, and FIG. 11E depicts the parameters of footprints.

In FIG. 11A, an example is shown where patient 50 is standing on the black step pathway for the evaluation and analysis of their footprints. In fact, performing evaluations and analyses while standing on the black step pathway is less challenging for patient 50 in terms of physical activity and daily functional abilities compared to wearing multiple sensors, especially for elderly individuals or patients with moderate to severe Parkinson's disease (PD).

Furthermore, the multi-spectrum image data of gait footprints provided by the black step pathway is reliable for analyzing the vertical alignment of the body's center of gravity and predicting the future risk of falls. Particularly, the analysis results of the lateral displacement and footprint analysis during forward and backward standing show a significant correlation with a series of falls.

FIG. 11B illustrates examples of different footprints in near-infrared imaging. These footprints may exhibit various patterns, which can be influenced by the balance of the body's center of gravity or disease-related factors, resulting in different patterns as shown or even more diverse patterns.

In FIG. 11C, the stability of patient 50's standing position (as shown in FIG. 7) is directly proportional to the Base of Support (BOS) and inversely proportional to the Center of Gravity (COG) height. Therefore, it is crucial to observe the footprints of the same patient 50 for assessing stability.

In FIG. 11D, the observation of these footprints is conducted by different patients 50 with or without assistive devices. In terms of stability evaluation in this embodiment, the footprint image area is divided into three “areas”: A, B, and C. The percentage of B/(A+B+C) is used as an observational index center.

Of course, the changes in the “area” of these three regions, A, B, and C, only provide initial evaluation and analysis for physical therapists. In clinical practice, artificial intelligence (AI) calculations are utilized to assist attending physicians in conducting evaluation and analysis and proposing corresponding exercise plans.

FIG. 11E illustrates the main parameters of single-foot footprints. In addition to the Foot Deviation Angle (L_{φ}), which represents the angle between the foot centerline and the same-side “walking straight line,” the parameters also include Forefoot Length (W1) and Forefoot Width (W2). The changes in the Foot Deviation Angle (L_{φ}) and the areas or contours of the footprints, such as Forefoot Length (W1) and Forefoot Width (W2), will result in clear “contrast images” in the near-infrared images. This allows easy identification of any “swelling” or “atrophy” in the single-foot footprints by patient 50 and physical therapists.

However, whether these footprints exhibit “swelling” or “atrophy” is only part of the preliminary evaluation and analysis for physical therapists. In clinical practice, AI calculations are used to assist attending physicians in conducting evaluation and analysis and proposing corresponding exercise plans.

Generally, maintaining a static posture, the Center of Pressure (COP) can reflect the position of the body's center of gravity. Therefore, it can be used as an indicator to evaluate balance ability (body control ability). The sway of the body's center of gravity, COP trajectory, and COP area are all factors in assessing balance ability. A shorter COP trajectory and smaller COP area indicate better balance ability.

Regarding the movement of COP trajectory, a comparison can be observed between standing in a parallel position and standing in a forward-backward position as shown in FIG. 8B. In the forward-backward standing position, there is greater lateral displacement, while in the parallel standing position, there is greater anteroposterior displacement. This is because the forward-backward standing position has a narrower base of support (BOS), resulting in greater lateral displacement.

In different conditions of eyes open and eyes closed, it can be observed that almost all parameters are larger when the eyes are closed compared to when the eyes are open. Under the same movement, the change in parameters after closing the eyes does not increase with age and exhibits irregular variations. Different directions of variation also have an impact under different standing positions. In the parallel standing position, there is greater anteroposterior displacement, while there is no difference in lateral displacement. In the forward-backward standing position, there is greater lateral displacement, but no difference in anteroposterior displacement.

From the above results, we can conclude that forward-backward standing is more difficult than parallel standing, and closed-eye condition is more challenging than eyes-open condition.

In everyday walking, normal individuals do not “think” about how to take each step. They can even chat, use their hands for other tasks, and adjust their gait and body posture according to various situations. However, patients with Parkinson's disease (PD), such as patient 50, do not experience such smoothness. Therefore, there is a need for further evaluation and analysis of gait disorders in both normal individuals and patients with PD.

Due to the characteristic flexed posture of patients with Parkinson's disease (PD), their center of gravity tends to shift forward. In order to maintain balance, patients with PD tend to take small and quick steps forward and “cannot” stop or change direction abruptly, resulting in a hurried gait. These characteristics can be easily identified from the multispectral imaging analysis of gait evaluation.

The complexity of the evaluation and analysis process is attributed to various reported clinical and non-clinical studies on different gait patterns. In clinical medicine, factors such as lower limb biomechanics and anatomy need to be considered, and a simple evaluation of gait based solely on footprints is insufficient.

To establish a certain degree of correlation between “image differences” and “center of gravity displacement,” our team has found that: (1) In the imaging analysis of moderate PD patients (FIG. 7C) after achieving stability through medication, the difficulty level is higher underwater compared to on land. (2) The difficulty level is higher for mild PD patients when their eyes are closed compared to when their eyes are open.

It can be inferred that there is a certain degree of correlation between these “image differences” and “center of gravity displacement,” which means that a correlation can be found between the “COP trajectory” and the corresponding movements.

In summary, the proposed evaluation and analysis and static pressure hydrotherapy exercise device aim to “assist” each patient in finding the most suitable mode and method for themselves.

As described above, for the footprints gait image evaluation and analysis method, a corresponding computer application (APP) software program will be developed in future patent applications to automatically assist in the analysis of more parameters. These may include important data analysis such as foot pressure intensity, duration of standing cessation phase, and duration of mental stress phase.

As mentioned above, in future patent applications, the footprints gait image evaluation and analysis method can be integrated with an intelligent remote healthcare system for frequent and automated risk evaluation and analysis using AI. This could be a feasible and cost-effective approach.

In summary, it is evident that this invention differs from the disclosed hardware (including the walkway) in Patent TWI757022 in terms of technical field and functionality.
(1) Definition and explanation of terms: (1A) Multispectral Definition: In this context, multispectral refers to three wavelength ranges: 8-14 μm for Far Infrared (FIR), 0.8-1.0 μm for Near Infrared (NIR), and 0.4-0.8 μm for Visible (VIR) light. However, in this invention, the term “multispectral imaging” refers to the images captured by the multispectral thermal imager 301. These include thermal images (also known as Far Infrared images) of the lower extremities of the patient (FIG. 1D), Near Infrared images of the footprints of the patient (FIGS. 1B, 1C, 7B, 8A, 8B, 8C), visible light images of the patient's entire body (FIG. 1A), and the underwater window image of the patient (FIG. 3A), which falls within the definition of visible light imaging.

Among these, the footprints on the black walkway captured in near-infrared images are primarily used for evaluation and analysis, with thermal and visible light images serving as supplementary information, providing clear and high-contrast near-infrared images.
(1B) Application examples of multispectral imaging in this invention: The differences in near-infrared images of the patient's footprints are correlated with the patient's trajectory, meaning that by analyzing the trajectory of the patient's center of gravity displacement and the differences in multispectral imaging, it is possible to identify the correlation between the patient's fall probability and abnormalities in lower extremity tissues.
(2) Technical Solutions:
(2-1) In contrast to the evaluation and analysis of patient gait footprints conducted solely on the ground in foreign studies, this invention provides an interactive comparison of multispectral images both on the black walkway surface and underwater. For example, the comparison can be performed from the ground to underwater and vice versa, with repeated interactive comparisons. After the repeated interactive comparisons, the optimal exercise method can be determined based on the compared images, and a subsequent exercise plan can be developed.
(2-2) Evaluating and analyzing gait solely on the ground is insufficient and cannot serve as a reliable predictor of falls for patients, especially elderly individuals. By considering the differences in step length and standing time variability of patients both on the ground and underwater, a clearer distinction can be made between fall-prone patients and non-fall-prone patients.
(2-3) By combining audiovisual guidance with the patient's own body sensation, the invention guides the patient through relevant hydrostatic water therapy exercises to improve their gait disorders.
(2-4) The evaluation and analysis of patient gait footprints provided by this invention can contribute to a better understanding of the gait patterns and help differentiate between pathological gait changes caused by specific exercise plans and compensatory gait changes.
(2-5) Although the invention does not provide a “treatment” for gait disorders, it can enhance the quality of life for patients and help control symptoms. In particular, physical therapists can use the invention to motivate patients to attempt exercises and activities that they may perceive as impossible.
(2-6) In other embodiments, the evaluation and analysis of gait disorders provided by this invention only serve as preliminary evaluations for patients. It does not replace further medical research, nor does it provide sufficient resources or equipment. The invention, based on general knowledge, offers initial evaluations to assist physical therapists in developing exercise plans and improving the patients' quality of life in their daily activities.
(3) Technical Features:
(3-1) The present invention relates to a hydrostatic water therapy system suitable for gait-impaired individuals, which includes an adjustable black walkway platform 100 and a hydrostatic water therapy pool 62.
(3-2) A multispectral thermal imager 301 is positioned at the lower end of the adjustable black walkway platform 100 to capture multispectral images of the gait of the gait-impaired individuals (patient 50) both inside and outside the hydrostatic water therapy pool 62. This feature distinguishes the invention from conventional hydrotherapy systems, which primarily focus on exercising in water environments, and from previous techniques or literature that analyze gait footprints solely on the ground.

For example, the present invention replaces the use of wearable sensors on the ground for gait analysis, as commonly employed in current methods such as those disclosed by the Industrial Technology Research Institute in Taiwan. Conventional hydrotherapy pools, such as HydroWork in the United States, primarily provide exercise functions. In contrast, the present invention not only offers exercise capabilities but also includes evaluation and analysis functions. For instance, while a patient in the hydrostatic water therapy pool 62 can practice tasks like carrying a teacup or putting on clothes, the system can simultaneously assess and analyze the patient's gait variations during these exercises to infer the correctness of their stance and the need for adjustment in COP (Center of Pressure) training, among other factors.

In summary, the hydrostatic water therapy system in this invention allows for the capture of multispectral images of the gait of the patient (gait-impaired individuals) both inside and outside the hydrostatic water therapy pool 62, facilitating the evaluation and analysis by a physical therapist and the development of corresponding exercise plans.
(3-3) In this hydrostatic water therapy system, a VC temperature-variable plate 80 is employed to provide clearer thermal images of the lower extremities of the patient (gait-impaired individuals) standing on the adjustable black walkway platform 100 to the multispectral thermal imager 301 within the system.
(3-4) The black walkway platform 20 in this hydrostatic water therapy system is designed to accommodate only one patient (gait-impaired individuals). This configuration ensures that the patient in the hydrostatic water therapy pool 62 is not affected by neighboring patients undergoing hydrostatic therapy in the warm water 61.
(3-5) The multispectral images captured by the multispectral thermal imager 301 of the patient (gait-impaired individuals) can be observed or recorded for further analysis.
(3-6) The black walkway platform 20 in this hydrostatic water therapy system incorporates near-infrared auxiliary light sources with a wavelength of 940 nm LEDs. This design ensures that the patient standing on the black walkway platform 20 remains focused and undistracted during the evaluation.
(3-7) A safety unit consisting of wide-base handrails, weight-reducing harnesses, and assistive devices can be placed on the black walkway platform 20 to provide comfort and alleviate fear for the patient (gait-impaired individuals).
(3-8) The present invention encompasses an evaluation and exercise method for gait-impaired individuals specifically designed for gait-impaired individuals. The evaluation method involves the capture of multispectral images from both the ground-based evaluation and analysis method 200 and the underwater evaluation and analysis method 300.
(3-9) The exercise method includes the exercise plans depicted in FIGS. 7B, 8, 8A, and 8B, which help determine the COP (Center of Pressure) movement trajectory of the patient (gait-impaired individuals). These exercise plans facilitate the implementation of corresponding hydrostatic therapy exercises.

This evaluation and exercise method for gait-impaired individuals, which captures multispectral images, allows for early diagnosis and helps identify the optimal exercise regimen for the patient (gait-impaired individuals).

The evaluation and exercise method for gait-impaired individuals includes the evaluation and exercise method for deviations in temporal and spatial parameters of gait-impaired individuals compared to normal gait, as depicted in FIGS. 7B, 8, 8A, and 8B.

### Patentability Discussion:

In order to establish a certain level of correlation between “image differences” and “falling probability,” as evident from the aforementioned exemplary figures and description, it is understood that both “image differences” and “falling probability” involve COP (Center of Pressure), which in turn relates to the body's center of gravity, COG (Center of Gravity), which affects the footprint area and outer contour.

Clearly, by utilizing the image differences in the footprints of patient 50, it is possible to predict the likelihood of falling.

This invention, after numerous trial experiments conducted by the inventors, explores various rational combinations and achieves an improvement level that surpasses the expectations of ordinary users of previous technical components such as thermal imagers, as well as prior patents such as Patent TWI666935 and Patent TWI425292. It is not something readily attainable by those skilled in the art and cannot be deemed as common knowledge in this technical field.

Although the invention has been disclosed and illustrated with reference to particular embodiments, the principles involved are susceptible for use in numerous other embodiments that will be apparent to persons skilled in the art. This invention is, therefore, to be limited only as indicated by the scope of the appended claims.

1. A hydrostatic water therapy system for gait-impaired individuals, comprising:
an adjustable black walkway platform, which includes adjustable pillars, a black walkway, a multispectral thermal imager, and a safety unit; and
a hydrostatic water therapy pool, which includes a pool body, a water source device, and a window;
wherein the hydrostatic water therapy system uses the adjustable black walkway platform and the hydrostatic water therapy pool to capture multispectral footprint images of the gait-impaired individual both inside and outside the pool, thereby achieving dual functionality of evaluation and corresponding exercise.

2. The hydrostatic water therapy system of claim 1, wherein the adjustable black walkway platform further includes a VC temperature control plate and a controller of the VC temperature control plate.

3. The hydrostatic water therapy system of claim 1, wherein the black walkway has a test area and a scale table on both sides.

4. The hydrostatic water therapy system of claim 1, wherein the water source device is configured to perform functions including: water circulation, replacement cleaning, disinfection sterilization, and hot and cold water supply and temperature control.

5. The hydrostatic water therapy system of claim 1, wherein the multispectral thermal imager includes a near-infrared auxiliary light source with 940 nm LEDs.

6. The hydrostatic water therapy system of claim 1, wherein the safety unit includes a wide-bottom handrail or a weight-reducing sling or a walker.

7. An evaluation and analysis system for gait-impaired individuals' footprints, which includes evaluation mode, evaluation method, evaluation subjects, evaluation items and images, visual and auditory guidance methods, and physical therapist coaching methods for evaluating the position of body center of gravity response, serving as the basis for taking relevant corresponding hydrostatic water therapy pool evaluation and analysis and exercise plan;
wherein the evaluation and analysis system is used to assess balance ability indicators of the gait-impaired individual's footprint COP area and movement trajectory, and assists in identifying the most effective exercise plan process.

8. The evaluation and analysis system of claim 7, wherein the images include far-infrared with a wavelength range of 8-14 μm, near-infrared with a wavelength range of 0.8-1.0 μm, and visible light with a wavelength range of 0.4-0.8 μm, all three types of footprint images.

9. The evaluation and analysis system of claim 7, wherein the position of the body center of gravity response further includes a connection line of the body's COP and COG.

10. The evaluation and analysis system of claim 7, wherein the evaluation and analysis system further includes a hydrostatic water therapy pool.

11. The evaluation and analysis system of claim 10, wherein the hydrostatic water therapy pool further includes warm water, a pool body, water source equipment, and a window.

12. The evaluation and analysis system of claim 11, wherein the material of the pool body is strengthened thick transparent glass.

13. The evaluation and analysis system of claim 8, wherein the visible light includes the window image of the pool body.
